SEQUENCE LISTING (12 sequences)

SEQ ID NO: 1 - protein, 315 aa, Homo sapiens
MEARAQSGNGSQPLLQTPRDGGRQRGEPDPRDALTQQVHVLSLDQIRAIRNTNEYTEGPTVVPRPGLKPAPRPSTQHKHERLHGLPEHRQPPRLQHSQVHSSARAPLSRSISTVSSGSRSSTRTSTSSSSSEQRLLGSSFSSGPVADGIIRVQPKSELKPGELKPLSKEDLGLHAYRCEDCGKCKCKECTYPRPLPSDWICDKQCLCSAQNVIDYGTCVCCVKGLFYHCSNDDEDNCADNPCSCSQSHCCTRWSAMGVMSLFLPCLWCYLPAKGCLKLCQGCYDRVNRPGCRCKNSNTVCCKVPTVPPRNFEKPT

SEQ ID NO: 2 - protein, 377 aa, Homo sapiens
MKFPGPLENQRLSFLLEKAITREAQMWKVNVRKMPSNQNVSPSQRDEVIQWLAKLKYQFNLYPETFALASSLLDRFLATVKAHPKYLSCIAISCFFLAAKTVEEDERIPVLKVLARDSFCGCSSSEILRMERIILDKLNWDLHTATPLDFLHIFHAIAVSTRPQLLFSLPKLSPSQHLAVLTKQLLHCMACNQLLQFRGSMLALAMVSLEMEKLIPDWLSLTIELLQKAQMDSSQLIHCRELVAHHLSTLQSSLPLNSVYVYRPLKHTLVTCDKGVFRLHPSSVPGPDFSKDNSKPEVPVRGTAAFYHHLPAASGCKQTSTKRKVEEMEVDDFYDGIKRLYNEDNVSENVGSVCGTDLSRQEGHASPCPPLQPVSVM

SEQ ID NO: 3 - protein, 347 aa, Homo sapiens
MCTKMEQPFYHDDSYTATGYGRAPGGLSLHDYKLLKPSLAVNLADPYRSLKAPGARGPGPEGGGGGSYFSGQGSDTGASLKLASSELERLIVPNSNGVITTTPTPPGQYFYPRGGGSGGGAGGAGGGVTEEQEGFADGFVKALDDLHKMNHVTPPNVSLGATGGPPAGPGGVYAGPEPPPVYTNLSSYSPASASSGGAGAAVGTGSSYPTTTISYLPHAPPFAGGHPAQLGLGRGASTFKEEPQTVPEARSRDATPPVSPINMEDQERIKVERKRLRNRLAATKCRKRKLERIARLEDKVKTLKAENAGLSSTAGLLREQVAQLKQKVMTHVSNGCQLLLGVKGHAF

SEQ ID NO: 4 - protein, 467 aa, Homo sapiens
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 5 - protein, 583 aa, Homo sapiens
MDLLPPKPKYNPLRNESLSSLEEGASGSTPPEELPSPSASSLGPILPPLPGDDSPTTLCSFFPRMSNLRLANPAGGRPGSKGEPGRAADDGEGIVGAAMPDSGPLPLLQDMNKLSGGGGRRTRVEGGQLGGEEWTRHGSFVNKPTRGWLHPNDKVMGPGVSYLVRYMGCVEVLQSMRALDFNTRTQVTREAISLVCEAVPGAKGATRRRKPCSRPLSSILGRSNLKFAGMPITLTVSTSSLNLMAADCKQIIANHHMQSISFASGGDPDTAEYVAYVAKDPVNQRACHILECPEGLAQDVISTIGQAFELRFKQYLRNPPKLVTPHDRMAGFDGSAWDEEEEEPPDHQYYNDFPGKEPPLGGVVDMRLREGAAPGAARPTAPNAQTPSHLGATLPVGQPVGGDPEVRKQMPPPPPCPGRELFDDPSYVNVQNLDKARQAVGGAGPPNPAINGSAPRDLFDMKPFEDALRVPPPPQSVSMAEQLRGEPWFHGKLSRREAEALLQLNGDFLVRESTTTPGQYVLTGLQSGQPKHLLLVDPEGVVRTKDHRFESVSHLISYHMDNHLPIISAGSELCLQQPVERKL

SEQ ID NO: 6 - protein, 718 aa, Homo sapiens
MEDLGENTMVLSTLRSLNNFISQRVEGGSGLDISTSAPGSLQMQYQQSMQLEERAEQIRSKSHLIQVEREKMQMELSHKRARVELERAASTSARNYEREVDRNQELLTRIRQLQEREAGAEEKMQEQLERNRQCQQNLDAASKRLREKEDSLAQAGETINALKGRISELQWSVMDQEMRVKRLESEKQELQEQLDLQHKKCQEANQKIQELQASQEARADHEQQIKDLEQKLSLQEQDAAIVKNMKSELVRLPRLERELKQLREESAHLREMRETNGLLQEELEGLQRKLGRQEKMQETLVGLELENERLLAKLQSWERLDQTMGLSIRTPEDLSRFVVELQQRELALKDKNSAVTSSARGLEKARQQLQEELRQVSGQLLEERKKRETHEALARRLQKRVLLLTKERDGMRAILGSYDSELTPAEYSPQLTRRMREAEDMVQKVHSHSAEMEAQLSQALEELGGQKQRADMLEMELKMLKSQSSSAEQSFLFSREEADTLRLKVEELEGERSRLEEEKRMLEAQLERRALQGDYDQSRTKVLHMSLNPTSVARQRLREDHSQLQAECERLRGLLRAMERGGTVPADLEAAAASLPSSKEVAELKKQVESAELKNQRLKEVFQTKIQEFRKACYTLTGYQIDITTENQYRLTSLYAEHPGDCLIFKATSPSGSKMQLLETEFSHTVGELIEVHLRRQDSIPAFLSSLTLELFSRQTVA

SEQ ID NO: 7 - protein, 222 aa, Homo sapiens
MAASVRQARSLLGVAATLAPGSRGYRARPPPRRRPGPRWPDPEDLLTPRWQLGPRYAAKQFARYGAASGVVPGSLWPSPEQLRELEAEEREWYPSLATMQESLRVKQLAEEQKRREREQHIAECMAKMPQMIVNWQQQQRENWEKAQADKERRARLQAEAQELLGYQVDPRSARFQELLQDLEKKERKRLKEEKQKRKKEARAAALAAAVAQDPAASGAPSS

SEQ ID NO: 8 - protein, 1972 aa, Homo sapiens
MGDDSEWLKLPVDQKCEHKLWKARLSGYEEALKIFQKIKDEKSPEWSKFLGLIKKFVTDSNAVVQLKGLEAALVYVENAHVAGKTTGEVVSGVVSKVFNQPKAKAKELGIEICLMYIEIEKGEAVQEELLKGLDNKNPKIIVACIETLRKALSEFGSKIILLKPIIKVLPKLFESREKAVRDEAKLIAVEIYRWIRDALRPPLQNINSVQLKELEEEWVKLPTSAPRPTRFLRSQQELEAKLEQQQSAGGDAEGGGDDGDEVPQIDAYELLEAVEILSKLPKDFYDKIEAKKWQERKEALESVEVLIKNPKLEAGDYADLVKALKKVVGKDTNVMLVALAAKCLTGLAVGLRKKFGQYAGHVVPTILEKFKEKKPQVVQALQEAIDAIFLTTTLQNISEDVLAVMDNKNPTIKQQTSLFIARSFRHCTASTLPKSLLKPFCAALLKHINDSAPEVRDAAFEALGTALKVVGEKAVNPFLADVDKLKLDKIKECSEKVELIHGKKAGLAADKKEFKPLPGRTAASGAAGDKDTKDISAPKPGPLKKAPAAKAGGPPKKGKPAAPGGAGNTGTKNKKGLETKEIVEPELSIEVCEEKASAVLPPTCIQLLDSSNWKERLACMEEFQKAVELMDRTEMPCQALVRMLAKKPGWKETNFQVMQMKLHIVALIAQKGNFSKTSAQVVLDGLVDKIGDVKCGNNAKEAMTAIAEACMLPWTAEQVVSMAFSQKNPKNQSETLNWLSNAIKEFGFSGLNVKAFISNVKTALAATNPAVRTAAITLLGVMYLYVGPSLRMFFEDEKPALLSQIDAEFEKMQGQSPPAPTRGISKHSTSGTDEGEDGDEPDDGSNDVVDLLPRTEISDKITSELVSKIGDKNWKIRKEGLDEVAGIINDAKFIQPNIGELPTALKGRLNDSNKILVQQTLNILQQLAVAMGPNIKQHVKNLGIPIITVLGDSKNNVRAAALATVNAWAEQTGMKEWLEGEDLSEELKKENPFLRQELLGWLAEKLPTLRSTPTDLILCVPHLYSCLEDRNGDVRKKAQDALPFFMMHLGYEKMAKATGKLKPTSKDQVLAMLEKAKVNMPAKPAPPTKATSKPMGGSAPAKFQPASAPAEDCISSSTEPKPDPKKAKAPGLSSKAKSAQGKKMPSKTSLKEDEDKSGPIFIVVPNGKEQRMKDEKGLKVLKWNFTTPRDEYIEQLKTQMSSCVAKWLQDEMFHSDFQHHNKALAVMVDHLESEKEGVIGCLDLILKWLTLRFFDTNTSVLMKALEYLKLLFTLLSEEEYHLTENEASSFIPYLVVKVGEPKDVIRKDVRAILNRMCLVYPASKMFPFIMEGTKSKNSKQRAECLEELGCLVESYGMNVCQPTPGKALKEIAVHIGDRDNAVRNAALNTIVTVYNVHGDQVFKLIGNLSEKDMSMLEERIKRSAKRPSAAPIKQVEEKPQRAQNISSNANMLRKGPAEDMSSKLNQARSMSGHPEAAQMVRREFQLDLDEIENDNGTVRCEMPELVQHKLDDIFEPVLIPEPKIRAVSPHFDDMHSNTASTINFIISQVASGDINTSIQALTQLFQIESLAREASTGVLKDLMHGLITLMLDSRIEDLEEGQQVIRSVNLLVVKVLEKSDQTNILSALLVLLQDSLLATASSPKFSELVMKCLWRMVRLLPDTINSINLDRILLDIHIFMKVFPKEKLKQCKSEFPIRTLKTLLHTLCKLKGPKILDHLTMIDNKNESELEAHLCRMMKHSMDQTGSKSDKETEKGASRIDEKSSKAKVNDFLAEIFKKIGSKENTKEGLAELYEYKKKYSDADIEPFLKNSSQFFQSYVERGLRVIEMEREGKGRISTSTGISPQMEVTCVPTPTSTVSSIGNTNGEEVGPSVYLERLKILRQRCGLDNTKQDDRPPLTSLLSKPAVPTVASSTDMLHSKLSQLRESREQHQHSDLDSNQTHSSGTVTSSSSTANIDDLKKRLERIKSSRK

SEQ ID NO: 9 - protein, 444 aa, Homo sapiens
MREIVHLQAGQCGNQIGAKFWEVISDEHGIDPTGTYHGDSDLQLERINVYYNEATGGNYVPRAVLVDLEPGTMDSVRSGPFGQIFRPDNFVFGQSGAGNNWAKGHYTEGAELVDAVLDVVRKEAESCDCLQGFQLTHSLGGGTGSGMGTLLISKIREEFPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQMFDAKNMMAACDPRHGRYLTVAAVFRGRMSMKEVDEQMLSVQSKNSSYFVEWIPNNVKTAVCDIPPRGLKMAATFIGNSTAIQELFKRISEQFTAMFRRKAFLHWYTGEGMDEMEFTEAESNMNDLVSEYQQYQDATAEEGEFEEEAEEEVA

SEQ ID NO: 10 - protein, 386 aa, Homo sapiens
MKDCSNGCSAECTGEGGSKEVVGTFKAKDLIVTPATILKEKPDPNNLVFGTVFTDHMLTVEWSSEFGWEKPHIKPLQNLSLHPGSSALHYAVELFEGLKAFRGVDNKIRLFQPNLNMDRMYRSAVRATLPVFDKEELLECIQQLVKLDQEWVPYSTSASLYIRPTFIGTEPSLGVKKPTKALLFVLLSPVGPYFSSGTFNPVSLWANPKYVRAWKGGTGDCKMGGNYGSSLFAQCEAVDNGCQQVLWLYGEDHQITEVGTMNLFLYWINEDGEEELATPPLDGIILPGVTRRCILDLAHQWGEFKVSERYLTMDDLTTALEGNRVREMFGSGTACVVCPVSDILYKGETIHIPTMENGPKLASRILSKLTDIQYGREESDWTIVLS

SEQ ID NO: 11 - protein, 840 aa, Homo sapiens
MNGEYRGRGFGRGRFQSWKRGRGGGNFSGKWREREHRPDLSKTTGKRTSEQTPQFLLSTKTPQSMQSTLDRFIPYKGWKLYFSEVYSDSSPLIEKIQAFEKFFTRHIDLYDKDEIERKGSILVDFKELTEGGEVTNLIPDIATELRDAPEKTLACMGLAIHQVLTKDLERHAAELQAQEGLSNDGETMVNVPHIHARVYNYEPLTQLKNVRANYYGKYIALRGTVVRVSNIKPLCTKMAFLCAACGEIQSFPLPDGKYSLPTKCPVPVCRGRSFTALRSSPLTVTMDWQSIKIQELMSDDQREAGRIPRTIECELVHDLVDSCVPGDTVTITGIVKVSNAEEGSRNKNDKCMFLLYIEANSISNSKGQKTKSSEDGCKHGMLMEFSLKDLYAIQEIQAEENLFKLIVNSLCPVIFGHELVKAGLALALFGGSQKYADDKNRIPIRGDPHILVVGDPGLGKSQMLQAACNVAPRGVYVCGNTTTTSGLTVTLSKDSSSGDFALEAGALVLGDQGICGIDEFDKMGNQHQALLEAMEQQSISLAKAGVVCSLPARTSIIAAANPVGGHYNKAKTVSENLKMGSALLSRFDLVFILLDTPNEHHDHLLSEHVIAIRAGKQRTISSATVARMNSQDSNTSVLEVVSEKPLSERLKVVPGETIDPIPHQLLRKYIGYARQYVYPRLSTEAARVLQDFYLELRKQSQRLNSSPITTRQLESLIRLTEARARLELREEATKEDAEDIVEIMKYSMLGTYSDEFGNLDFERSQHGSGMSNRSTAKRFISALNNVAERTYNNIFQFHQLRQIAKELNIQVADFENFIGSLNDQGYLLKKGPKVYQLQTM

SEQ ID NO: 12 - protein, 750 aa, Homo sapiens
MMEELHSLDPRRQELLEARFTGVGVSKGPLNSESSNQSLCSVGSLSDKEVETPEKKQNDQRNRKRKAEPYETSQGKGTPRGHKISDYFEFAGGSAPGTSPGRSVPPVARSSPQHSLSNPLPRRVEQPLYGLDGSAAKEATEEQSALPTLMSVMLAKPRLDTEQLAQRGAGLCFTFVSAQQNSPSSTGSGNTEHSCSSQKQISIQHRQTQSDLTIEKISALENSKNSDLEKKEGRIDDLLRANCDLRRQIDEQQKMLEKYKERLNRCVTMSKKLLIEKSKQEKMACRDKSMQDRLRLGHFTTVRHGASFTEQWTDGYAFQNLIKQQERINSQREEIERQRKMLAKRKPPAMGQAPPATNEQKQRKSKTNGAENETLTLAEYHEQEEIFKLRLGHLKKEEAEIQAELERLERVRNLHIRELKRIHNEDNSQFKDHPTLNDRYLLLHLLGRGGFSEVYKAFDLTEQRYVAVKIHQLNKNWRDEKKENYHKHACREYRIHKELDHPRIVKLYDYFSLDTDSFCTVLEYCEGNDLDFYLKQHKLMSEKEARSIIMQIVNALKYLNEIKPPIIHYDLKPGNILLVNGTACGEIKITDFGLSKIMDDDSYNSVDGMELTSQGAGTYWYLPPECFVVGKEPPKISNKVDVWSVGVIFYQCLYGRKPFGHNQSQQDILQENTILKATEVQFPPKPVVTPEAKAFIRRCLAYRKEDRIDVQQLACDPYLLPHIRKSVSTSSPAGAAIASTSGASNNSSSN

(19) United States
(12) Patent Application Publication
(10) Pub. No.: US 2012/0157342 A1
(43) Pub. Date: Jun. 21, 2012

(54) PREDICTIVE BIOMARKERS USEFUL FOR CANCER THERAPY MEDIATED BY A WEE1 INHIBITOR

(76) Inventors: Shinji Mizuarai, Ibaraki (JP); Hiraku Itadani, Ibaraki (JP); Kazunori Yamanaka, Osaka (JP); Toshihide Nishibata, Ibaraki (JP); Tsuyoshi Arai, Shizuoka (JP); Hiroshi Hirai, Ibaraki (JP); Hidehito Kotani, Ibaraki (JP)

(21) Appl. No.: 13/393,915

(22) PCT Filed: Aug. 26, 2010
(86) PCT No.: PCT/JP2010/064976

§ 371 Date: Mar. 2, 2012

Related U.S. Application Data
(60) Provisional application No. 61/239,104, filed on Sep. 2, 2009.

(51) Int. Cl. C12Q 1/68; C40B 30/04

(52) U.S. Cl. 506/9; 435/6.11

(57) ABSTRACT

The present invention provides the identification of biomarker gene sets whose expression levels are useful for predicting a patient's response to a therapeutically effective dose of a Wee1 inhibitor as well the ability to predict said response prior to dosing with the Wee1 inhibitor. Additional uses are also disclosed in the specification.

### TECHNICAL FIELD

The present invention relates generally to the identification of potential responder biomarker gene set(s) whose expression levels are useful for predicting a patient's response to treatment with an anti-proliferative agent, particularly one that is responsive to a Wee1 inhibitor. More, the invention provides a clinician with the tools necessary to predict a patient's potential response to treatment an anti-proliferative agent such as a Wee1 inhibitor. In certain embodiments, the invention provides a skilled artisan with the means to identify whether a patient presenting with a cancerous condition, in particular, a condition mediated by a dysfunctional or aberrant p53 is likely to respond to treatment with a Wee1 inhibitor, prior to dosing with the Wee1 inhibitor. The ability to screen a potential patient's sensitivity to treatment with a Wee1 inhibitor pre-dose via utilizing methods of the invention, e.g., by quantifying biomarker expression prior to administering the Wee1 inhibitor, is an advantage over current treatment standards because it not only allows for early intervention but it also prevents subjecting a patient to the unnecessary side effects of treatment with an anti-cancer agent.

As well, it provides the clinician with important information about a patient's potential response at an earlier stage of the treatment protocol and in those rare cases where the data read out relative to the biomarker signature detailed herein suggests that the patient is unlikely to respond, it prevents the subject from incurring any additional discomfort and or side effects related to treatment with the particular anti-cancer agent.

### BACKGROUND ART

For successfully implementing cancer control in individuals presenting with cancer or suspected of having an increased risk of developing cancer, a mechanism must exist that rapidly predicts efficacy of potential agents used in clinical chemoprevention trials or medical treatments. A traditional approach to evaluating such agents generally relies on the use of laboratory animal models as a standard, often with a reduction in tumor incidence or burden being the accepted measurement of the therapeutic efficacy of the agent. Attention is now being drawn to the use of biomarkers for purposes of determining the usefulness or the efficacy of potential cancer therapeutics.

This so called “personalized medicine,” is expected to reduce the treatment burden on the patient as well as contribute to an overall reduction in the medical cost of the treatment. As well, it is expected to reduce undesirable side effects attendant the treatment.

Furthermore, the availability of the biomarkers makes them useful in analyzing, at a molecular level, the disease-related genes of an individual, which ultimately will allow for a more personalized approach in treating individual patients or sub-population of patients sharing similar traits, with fewer complications and side-effects.

Wee1 is a serine/threonine kinase regulating G2/M cell cycle checkpoint. In p53 negative tumors in which G1 cell cycle checkpoint is compromised, DNA damaging agent causes cell cycle arrest at G2/M phase in cancer cells. Thus, the abrogation of G2/M cell cycle checkpoint by Wee1 inhibition specifically causes cell death in p53 negative tumor. The concept for Wee1 inhibitor is to selectively sensitize p53-deficient tumors to different chemotherapies without increasing the chemotherapy-related toxicity to normal tissues.

In the early clinical development of anti-cancer agents, clinical trials are typically designed to evaluate the efficacy of agents, and pharmacokinetics, as well as to identify a suitable dose and schedule for further clinical evaluation. Scientists believe that the development of new validated indicators will lead to significant reductions in healthcare and drug development costs as well as provide a tool for achieving successful preventive intervention. Increasingly, efforts are being expended towards identifying high-risk individuals, who are at risk of or susceptible of resistant to a particular therapeutic moiety or alternatively, not responding to a particular therapeutic moiety. Earlier identification of such at-risk patients before administrating agents would help in the development of molecular-targeted interventions to prevent or delay neoplasia. Mindful that prediction of responses are necessary for the selection of neoadjuvant or adjuvant chemotherapy, it would be useful to be able to identify clinically relevant indicators, which may predict not only the final outcome of a chemopreventive trial but also help identify high-risk patients. After all, avoiding ineffective therapies is as important as identifying effective ones.

As a consequence, a great deal of effort is being directed to using new technologies to find new classes of biomarkers, which is becoming one of the highly prized goals of cancer research. See Petricoin et al., 2002, Nature Reviews Drug Discovery, 1:683-695; Sidransky, 2002, Nature Reviews Cancer, 2:210-219. Overall, risk biomarkers will find use not only in diagnosis but also confirm response to therapy, identify candidates who may best be suited for a particular chemopreventive intervention, aid in the rational design of future intervention therapy. The study of biomarkers that can possibly confirm how a person's disease may progress or respond to treatment, falls under the category of chemoprevention. Biomarkers used to predict a response to an intervention are called responder biomarkers (Science. 2006 May 26; 312(5777):1165-8). Examples of biomarkers include genetic markers (e.g., nuclear aberrations [such as micronuclei], gene amplification, and mutation), gene-expression markers (e.g., mRNA up- or down-regulation), protein marker (e.g., up- or down-regulation, phosphorylation of protein of interest) cellular markers (e.g., differentiation markers and measures of proliferation, such as thymidine labeling index), histologic markers (e.g., premalignant lesions, such as leukoplakia and colonic polyps), and biochemical and pharmacologic markers (e.g., ornithine decarboxylase activity, and radiology imaging reagents).

The identification of these biomarkers may be carried out by analyzing changes in specific polypeptides, metabolites or mRNAs, as predicted by the known biology associated with the molecule targeted by the agent of interest. Alternatively, biomarkers can be identified by analyzing global changes in polypeptides or mRNA in cells or tissues exposed to efficacious doses of the agent. Once identified, these biomarkers can be used to tailor a patient's clinical protocol, such as, by example, being able to predict a patient's response to a particular treatment protocol with a particular therapeutic moiety. As well, the pre-dose expression levels of at least one or more of the biomarkers detailed herein will also find use in predicting a patient's response to a Wee1 inhibitor, as disclosed herein, and based upon the readout, followed by treatment with a Wee1 inhibitor.

The art recognizes that the phosphorylation levels or state of Tyr15 residue of CDC2, which is a substrate of Wee1, provides a way to measure response to a Wee1 or its inhibitor. See Wang et al., cancer res. 61: 8211-8217. However, this particular marker fails to provide a quantitative measurement with which to quantify a response. In addition, the assay requires large amounts of the biopsy samples to assay for the phosphorylated—CDC2 assay.

Means of analyzing response to Wee1 or a Wee1 inhibitor by means of a specific biomarker has also failed to provide the status of p53. (Cancer Biol Ther. March; 3(3):305-13 (2004)) As well, the sensitivity to Wee1 inhibitor is variable among p53 negative cancers. Therefore, the unpredictability in the prior art and p53 in general prompted the inventors to seek to identify hyper-responders among p53 negative tumors as a means of increasing responder rates relative to Wee1 inhibitors.

### DISCLOSURE OF THE INVENTION

The present invention relates generally to the identification of biomarker gene set(s) whose expression level(s) are useful for predicting a patients response to a cancer therapy treatment comprising a Wee1 inhibitor, as well as determining/predicting a patients response, e.g., sensitivity or resistance to an anti-cancer agent such as a Wee1 inhibitor, based upon a read out of a biomarker signature in said patient prior to exposure to the Wee1 inhibitor.

The present invention also relates generally to a method for predicting a patient's response to treatment with a Wee1 inhibitor by determining, for example, expression level(s) of one or more of the biomarker gene or gene set(s) disclosed herein.

The invention further provides the above method(s), applied to predicting patients as having good response or poor response relative to treatment with a Wee1 inhibitor. For the Wee1 gene markers, the invention provides that the method may be used wherein the plurality of genes is at least 5, or 10 or 12 of the Wee1 markers listed in Table 1. In certain embodiments, the optimum 12 markers listed in Table 1 are used. In yet other aspects, at least one or more of the gene markers listed in Table 1 are used. Likewise, for pre-administration prediction purposes, the invention provides that at least 1, 5, 7, 10 or 12 gene markers listed in Table 1 may be used.

The present invention further relates to a method for treating a patient with a Wee1 inhibitor, using the results obtained from the prediction of a patient's response to treatment with a Wee1 inhibitor.

In another aspect, the invention describes the link between particular biomarker genes detailed herein and a Wee1 inhibitor. Towards this end, the specification demonstrates that expression of at least one or more of the biomarker genes in cancer cells are altered between high-sensitivity cells and low-sensitivity cells to a Wee1 inhibitor. This, in turn, demonstrates their utility as potential diagnostic and prediction biomarkers relative to the use of a Wee1 inhibitor.

In one embodiment of the present invention, the biomarkers comprises one or more genes whose expression levels were altered between high-sensitivity cells and low-sensitivity cells to an anti-cancer agent, e.g., Wee1 inhibitor.

In yet another embodiment, the biomarkers comprises at least one biomarker gene or its gene product selected from the group consisting of SPRY2, CCNI, JUNB, SMAD2, SHC1, MAD1L1, GADD45GIP1, CKAP5, TUBB4, BCAT1, MCM8 and TLK2. See Table 1.

Table 1 lists one variant of each biomarker genes as a representative example thereof, but any variant of each biomarker gene can be equally used for the purpose of the present invention.

### BRIEF DESCRIPTION OF DRAWINGS

FIG. 1 shows sensitivity of 22 NSCLC cell lines with p53 deficient to Cisplatin/Compound A as bliss additivism index.

FIG. 2-1 and FIG. 2-2 show hierarchical clustering analysis of up-/down-regulated genes in p53 deficient 22 NSCLC cell lines treated with Cisplatin/Compound A.

FIG. 3 shows the result of leave-one-out-cross-validation test of the 134 genes which shows high prediction accuracy for the sensitivity of Cisplatin/Compound A combination treatment.

FIG. 4 shows the result of leave-one-out-cross-validation test of the 134 genes. Hyper-responder signature genes (134) predicts the sensitivity to both Compound A/Carboplatin and Compound A/Gemcitabine combinations treatment

FIG. 5 shows expression ratio of MAD1 and SMAD in hyper-responder and normal-responder p53 deficient NSCLC cell lines treated with Gemcitabine/Compound A, Carboplatin/Compound A and Cisplatin/Compound A.

### DETAILED DESCRIPTION OF THE INVENTION

As recognized by various cancer researchers, it is becoming very important to identify potential responder biomarker(s) useful in predicting the therapeutic efficacy of an anti-cancer agent, e.g., Wee1 inhibitor particularly in a clinical trial and treatment. Analysis of expression responder biomarker(s) are considered to be more feasible and less burdensome for patients, because the required samples for the analysis are smaller compared with conventional biomarker including detection of protein phosphorylation with immunohistochemistry or DNA sequencing to detect genetic alteration.

The present invention relies on the surprising discovery of identifying responder biomarker(s) for a Wee1 inhibitor, which will have utility in predicting a patient's response to a treatment protocol comprising a Wee1 inhibitor.

After conducting vigorous studies, the inventors found that mRNA expression of at least one or more genes, preferably a set of genes (Wee 1 responder gene signature) as disclosed herein was specifically altered, e.g., increased or decreased in biological samples. The altered expression or change in expression was observed in lung cancers. The gene expression responder marker(s) of the present invention is quantifiable versus previously known responder marker(s) which measures the status of p53. As well, the identification and development of gene expression responder marker(s) disclosed herein is advantageous over the previously identified biomarker, supra, in that it is less time consuming and thus cheaper and also requires small amount of biopsied tissue.

### DEFINITIONS

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.”

“Wee1 inhibitor” means any compound or agent that inhibits the activity of one or more Wee1 proteins. The compound or agent may inhibit Wee1 activity by direct or indirect interaction with Wee1 protein or it may activity act to prevent expression of one or more Wee1 gene. Small molecule Wee1 inhibitors are described, for example, in US2005/0250836 patent publication, WO2003/091255 patent publication, Cancer Research vol. 61, pp 8211-8217 and Bioorg & Med. Chem. Lett. Vol. 15, pp. 1931-1935, and other Wee1 inhibitors are described, for example, in WO99/61444 and WO2004/041823 patent publications, but it does not limited to them.

“Gene marker” or “marker” means an entire gene, or a portion thereof, such as an EST derived from that gene, the expression or level of which changes between certain conditions. Where the expression of the gene correlates with a certain condition, for example a drug treatment or a disease state, the gene is a marker for that condition.

“Marker-derived polynucleotides” means the RNA transcribed from a marker gene, any cDNA or cRNA produced therefrom, and any nucleic acid derived therefrom, such as synthetic nucleic acid having a sequence derived from the gene corresponding to the marker gene.

A “similarity value” is a number that represents the degree of similarity between two things being compared. For example, a similarity value may be a number that indicates the overall similarity between a patient's expression profile using specific phenotype-related markers and a control specific to that phenotype (for instance, the similarity to a “good prognosis” template, where the phenotype is a good prognosis). The similarity value may be expressed as a similarity metric, such as a correlation coefficient, or may simply be expressed as the expression level difference, or the aggregate of the expression level differences, between a patient sample and a template.

As used herein, the terms “measuring expression levels,” “obtaining an expression level” and the like, includes methods that quantify target gene expression level exemplified by a transcript of a gene, including microRNA (miRNA) or a protein encoded by a gene, as well as methods that determine whether a gene of interest is expressed at all. Thus, an assay which provides a “yes” or “no” result without necessarily providing quantification of an amount of expression is an assay that “measures expression” as that term is used herein. Alternatively, the term may include quantifying expression level of the target gene expressed in a quantitative value, for example, a fold-change in expression, up or down, relative to a control gene or relative to the same gene in another sample, or a log ratio of expression, or any visual representation thereof, such as, for example, a “heatmap” where a color intensity is representative of the amount of gene expression detected. Exemplary methods for detecting the level of expression of a gene include, but are not limited to, Northern blotting, dot or slot blots, reporter gene matrix (see, for example, U.S. Pat. No. 5,569,588), nuclease protection, RT-PCR, microarray profiling, differential display, SAGE (Velculescu et al., 1995, Science 270:484-87), Digital Gene Expression System (see WO2007076128; WO2007076129), multiplex mRNA assay (Tian et al., 2004 Nucleic Acids Res. 32:e126), PMAGE (Kim et al., 2007 Science 316:1481-84), cDNA-mediated annealing, selection, extension and ligation assay (DASL, Bibikova, et al., 2004, AJP 165:1799-807), multiplex branched DNA assay (Flagella et al., 2006, Anal. Biochem. 352:50-60), 2D gel electrophoresis, SELDI-TOF, ICAT, enzyme assay, antibody assay, and the like.

As used herein, “subject” refers to an organism or to a cell sample, tissue sample or organ sample derived therefrom, including, for example, cultured cell lines, biopsy, blood sample or fluid sample containing a cell. In many instances, the subject or sample derived therefrom, comprises a plurality of cell types. In one embodiment, the sample includes, for example, a mixture of tumor cells and normal cells. In one embodiment, the sample comprises at least 10%, 15%, 20%, et seq., 90%, or 95% tumor cells. In one embodiment, the organism is a mammal, such as a human, canine, murine, feline, bovine, ovine, swine or caprine. In a particular embodiment, the organism is a human patient.

“Patient” as that term is used herein, refers to the recipient in need of medical intervention or treatment. Mammalian and non-mammalian patients are included. In one embodiment, the patient is a mammal, such as a human, canine, murine, feline, bovine, ovine, swine or caprine. In a particular embodiment, the patient is a human.

The term “treating” in its various grammatical forms in relation to the present invention refers to preventing (i.e. chemoprevention), curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

“Treatment of cancer”, as used herein, refers to partially or totally inhibiting, delaying or preventing the progression of cancer including cancer metastasis; inhibiting, delaying or preventing the recurrence of cancer including cancer metastasis; or preventing the onset or development of cancer (chemoprevention) in a mammal, for example a human. In addition, the methods of the present invention may be practiced for the treatment of chemoprevention of human patients with cancer. However, it is also likely that the methods would also be effective in the treatment of cancer in other mammals.

As used herein, the term “therapeutically effective amount” is intended to qualify the amount of the treatment in a therapeutic regimen necessary to treat cancer. This includes combination therapy involving the use of multiple therapeutic agents, such as a combined amount of a first and second treatment where the combined amount will achieve the desired biological response. The desired biological response is partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human.

As used herein, the terms “combination treatment”, “combination therapy”, “combined treatment” or “combinatorial treatment”, used interchangeably, refer to a treatment of an individual with at least two different therapeutic agents. According to the invention, the individual is treated with a first therapeutic agent, preferably a DNA damaging agent and/or a Wee 1 inhibitor as described herein. The second therapeutic agent may be another Wee1 inhibitor or may be any clinically established anti-cancer agent as defined herein. A combinatorial treatment may include a third or even further therapeutic agent.

“Status” means a state of gene expression of a set of genetic markers whose expression is strongly correlated with a particular phenotype. For example, “p53 status” means a state of gene expression of a set of genetic markers whose expression is strongly correlated with that of p53 gene, wherein the pattern of these genes' expression differs detectably between tumors expressing the protein and tumors not expressing the protein.

“Good prognosis” means that a patient is expected to have no distant metastases of a tumor within five years of initial diagnosis of cancer.

“Poor prognosis” means that a patient is expected to have distant metastases of a tumor within five years of initial diagnosis of cancer.

### Embodiment(s) of the Invention

A broad aspect of the invention concerns the identification of at least one or more biomarker genes whose expression is correlated with a response to a Wee1 inhibitor. Table 1 lists one such gene marker set (signature) whose expression level(s) are correlated with a response to an anti-cancer agent such as Wee1 inhibitor. The expression levels in response to the inhibitor may change relative to the entire gene set or just one gene from the gene set or a combination of different genes selected from Table 1.

Methods of using some or all of the marker genes detailed in Table 1 to predict a subject's sensitivity or resistance to a Wee1 inhibitor are also provided as are methods for determining whether a subject needs to exposed to a Wee1 inhibitor, or predict whether treatment with a cancer therapeutic agent, particularly Wee1 inhibitor will be effective.

### A. Classification of a Cell Sample Having Sensitivity to Wee1 Inhibitor

(i) Identification of Markers

The present invention provides gene biomarkers whose expression co-relates with a response to a Wee1 inhibitor. Generally, the marker sets were identified as detailed in the Examples set forth below by determining which of the numerous genes had expression patters that correlated with the conditions or indications.

(ii) Sample Collection

In the present invention, target polynucleotide molecules are extracted from a sample taken from an individual afflicted with a cancer. The sample may be collected in any clinically acceptable manner, but must be collected such that marker-derived polynucleotides (i.e., RNA) are preserved. mRNA or nucleic acids derived therefrom (i.e., cDNA or amplified DNA) are preferably labeled distinguishably from standard or control polynucleotide molecules, and both are simultaneously or independently hybridized to a microarray comprising some or all of the markers or marker sets or subsets described above. Alternatively, mRNA or nucleic acids derived therefrom may be labeled with the same label as the standard or control polynucleotide molecules, wherein the intensity of hybridization of each at a particular probe is compared. A sample may comprise any clinically relevant tissue sample, such as a tumor biopsy or fine needle aspirate, or a sample of bodily fluid, such as blood, plasma, serum, lymph, ascitic fluid, cystic fluid, urine or nipple exudate. The sample may be taken from a human, or, in a veterinary context, from non-human animals such as ruminants, horses, swine or sheep, or from domestic companion animals such as felines and canines.

Methods for preparing total and poly(A)+RNA are well known and are described generally in Sambrook et al., MOLECULAR CLONING—A LABORATORY MANUAL (2ND ED.), Vols. 1 3, Cold Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)).

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294 5299 (1979)). Poly(A)+RNA is selected by selection with oligo-dT cellulose (see Sambrook et al., MOLECULAR CLONING—A LABORATORY MANUAL (2ND ED.), Vols. 1 3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol. If desired, RNAse inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA). Most mRNAs contain a poly(A) tail at their 3′ end. This allows them to be enriched by affinity chromatography, for example, using oligo(dT) or poly(U) coupled to a solid support, such as cellulose or SEPHADEX™ medium (see Ausubel et al., CURRENT PROTOCOLS IN MOLECULAR BIOLOGY, vol. 2, Current Protocols Publishing, New York (1994). Once bound, poly(A)+mRNA is eluted from the affinity column using 2 mM EDTA/0.1% SDS.

The sample of RNA can comprise a plurality of different mRNA molecules, each different mRNA molecule having a different nucleotide sequence. In a specific embodiment, the mRNA molecules in the RNA sample comprise at least 100 different nucleotide sequences. More preferably, the mRNA molecules of the RNA sample comprise mRNA molecules corresponding to each of the marker genes. In another specific embodiment, the RNA sample is a mammalian RNA sample.

In a specific embodiment, total RNA or mRNA from cells are used in the methods of the invention. The source of the RNA can be cells of a plant or animal, human, mammal, primate, non-human animal, dog, cat, mouse, rat, bird, yeast, eukaryote, prokaryote, etc. In specific embodiments, the method of the invention is used with a sample containing total mRNA or total RNA from 1.times.10.sup.6 cells or less. In another embodiment, proteins can be isolated from the foregoing sources, by methods known in the art, for use in expression analysis at the protein level.

Probes to the homologs of the marker sequences disclosed herein can be employed preferably wherein non-human nucleic acid is being assayed.

(iii) Prediction of Sensitivity and/or Resistance of a Cell Sample to Wee1 Inhibitor Treatment

The invention provides a set of 12 genetic markers whose expression is correlated with a subject's response to a treatment with a Wee1 inhibitor. A set of these markers identified as useful for diagnosis or pre-dose prediction is listed in Table 1—SEQ ID Nos. 1-12. The invention also provides a method of using these markers to distinguish tumor types in diagnosis or pre-dose prediction.

Any of the sets of markers provided above may be used alone specifically or in combination with markers outside the set. Any of the marker sets provided above may also be used in combination with other markers for Wee1 mediated disorders such as cancer, or for any other clinical or physiological condition.

In one aspect, the present invention provides a set of gene markers (Table 1) that can be used to predict a cell sample as having sensitivity to a biologically active dose of a Wee1 inhibitor. In some instances it is of value to determine if a particular cell population has sensitivity or resistance to a therapeutic dose of a Wee1 inhibitor. In one embodiment, the markers are listed in Table 1.

The invention also provides subsets of at least 1, 2, 3, 4, or 5 markers derived from the set listed in Table 1. The invention further provides a set of markers derived from Table 1 that are optimal for predicting a patient's response to a treatment protocol comprising a Wee1 inhibitor.

TABLE 1
Up-/Down regulated genes that are functionally related to
Wee1 or G2/M cell cycle among signature genes

| | | | Change |
|---|---|---|---|
| Accession | | | Down-regulated/ |
| Number | Gene Symbol | SEQ ID NO: | up-regulated |
| NP_005833 | SPRY2 | SEQ ID NO: 1 | Up-regulated |
| NP_006826 | CCNI | SEQ ID NO: 2 | Down-regulated |
| NP_002220 | JUNB | SEQ ID NO: 3 | Down-regulated |
| NP_005892 | SMAD2 | SEQ ID NO: 4 | Down-regulated |
| NP_892113 | SHC1 | SEQ ID NO: 5 | Down-regulated |
| NP_003541 | MAD1L1 | SEQ ID NO: 6 | Up-regulated |
| NP_443082 | GADD45GIP1 | SEQ ID NO: 7 | Up-regulated |
| NP_055571 | CKAP5 | SEQ ID NO: 8 | Up-regulated |
| NP_006078 | TUBB4 | SEQ ID NO: 9 | Up-regulated |
| NP_005495 | BCAT1 | SEQ ID NO: 10 | Up-regulated |
| NP_115874 | MCM8 | SEQ ID NO: 11 | Up-regulated |
| NP_006843 | TLK2 | SEQ ID NO: 12 | Up-regulated |

In one embodiment, the selected markers are selected from Table 1 to include at least 1, 2 or 3 up-regulated genetic markers and at least 1, 2 or 3 down-regulated genetic markers. In other embodiments, the selected markers are all up-regulated markers or all down-regulated markers relative to a reference expression level.

In some embodiments, the markers are selected from Table 1 based upon a pre-determined threshold, wherein the pre-determined threshold is based upon marker gene expression measurements taken in control samples exposed to a Wee1 inhibitor. The pre-determined threshold may be expressed in several way, including, but not limited to, a fold change, up or down, of 1.2-fold change or greater, 1.3-fold or greater or 1.4-fold or greater, or 1.5-fold or greater, 1.6-fold or greater, 1.7-fold or greater, 1.8-fold or greater, 1.9-fold or greater, 2.0-fold or greater, or 3.0-fold or greater. The 2-fold means 2-fold up-regulated or ½-fold down-regulated of the markers in Wee1 inhibitor treated samples compared with non-treated control samples.

In another aspect of the invention, gene markers and methods are provided that are useful in predicting sensitivity and/or resistance of a subject to treatment with a Wee1 inhibitor. In one embodiment of this aspect of the invention, the gene markers or subset thereof, are used to make a drug response prediction based upon gene expression levels measured in a cell sample comprising tumor cells before Wee1 inhibitor treatment. The Wee1 response prediction markers are listed in Table 1. Table 1 lists gene markers, SPRY2, CCNI, JUNB, SMAD2, SHC1, MAD1L1, GADD45GIP1, CKAP5, TUBB4, BCAT1, MCM8 and TLK2, whose expression levels are correlated with sensitivity of cells to Wee1 inhibitor treatment.

In one embodiment of this aspect of the invention, Wee1 inhibitor sensitivity or resistance is predicted in a subject using 2 or more gene markers selected from Table 1. In another embodiment, sensitivity or resistance are predicted in a subject using at least 1, 2, 3, 4 or 5 markers selected from Table 1. One aspect of the present invention provides a method of using these sets of Wee1 inhibitor biomarkers to predict whether a subject with cancer will respond to treatment with a Wee1 inhibitor.

In certain embodiments, the invention comprises using data obtained from the biomarker predictor set as a means of determining whether a patient should continue treatment with a We1 inhibitor or be treated with a Wee1 inhibitor in the first place. Thus, patients exhibiting a favorable data set, e.g., a sensitivity signature, may continue treatment with the Wee1 inhibitor or start treatment with a Wee1 inhibitor. The methods of the invention may also be used to stratify patient population into a treatment group, e.g., those that can be treated with a Wee1 inhibitor and thus may be enrolled into a therapeutic regiment employing a Wee1 inhibitor or a non-treatment group, e.g., those that are not amenable to treatment with a Wee1 inhibitor. Towards this end, the methods of the invention may also be used to identify patients who may need to be pulled out of therapeutic protocol comprising a Wee1 inhibitor where the biomarker signature is not positive, e.g., non-sensitive signature.

In another embodiment, the method comprising:

(a) calculating a measure of similarity between a first expression profile and a Wee1 inhibitor sensitivity (responder) template, or calculating a first measure of similarity between said first expression profile and said Wee1 inhibitor sensitivity (responder) template and a second measure of similarity between said first expression profile and a Wee1 inhibitor resistance (non-responder) template, said first expression profile comprising a measured expression level of a gene in a cell sample obtained from said subject, wherein said cell sample comprises cancer cells and is obtained from said subject prior to treatment of said subject with a Wee1 inhibitor, said Wee1 inhibitor sensitivity (responder) template comprising an expression level of said gene that is average expression level of the gene in a first plurality of control cell samples that are sensitive to treatment with said Wee1 inhibitor, and said Wee1 inhibitor resistance (non-responder) template comprising an expression level of said gene that is average expression level of the gene in a second plurality of control cell samples that are resistant to treatment with said Wee1 inhibitor, said first plurality of genes consisting of at least 1 or more, preferably 2 or more of the genes for which markers are listed in Tables 1;

(b) predicting that said subject will:

(i) be sensitive to Wee1 inhibitor treatment if said first expression profile has a high similarity to said Wee1 inhibitor sensitivity (responder) template or has a higher similarity to said Wee1 inhibitor sensitivity (responder) template than to said Wee1 inhibitor resistance (non-responder) template, or

(ii) be resistant to Wee1 inhibitor treatment if said first expression profile has a low similarity to said Wee1 inhibitor sensitivity (responder) template or has a higher similarity to said Wee1 inhibitor resistance (non-responder) template than to said Wee1 inhibitor sensitivity (responder) template;

wherein said first expression profile has a high similarity to said Wee1 inhibitor sensitivity (responder) template if the similarity to said Wee1 inhibitor sensitivity (responder) template is above a predetermined threshold, or has a low similarity to said Wee1 inhibitor sensitivity (responder) template if the similarity to said Wee1 inhibitor sensitivity (responder) template is below said predetermined threshold. The method further proposes treating the patient with a Wee1 inhibitor based upon the prediction, e.g., treating patients demonstrating a sensitive profile and pulling out patients from a treatment protocol comprising a Wee1 inhibitor if their signature profile is that of a non-responder, e.g., non-responsive to Wee1 inhibitor. Similarity Between a Gene Expression Profile and a Sensitivity/Resistance Template.

The degree of similarity between a gene expression profile obtained from a cellular sample and a template profile can be determined using any method known in the art. For example, Dai et al., describe a number of different ways of calculating gene expression templates and corresponding gene marker gene sets useful in classifying breast cancer patients (U.S. Pat. No. 7,171,311; WO2002103320; WO2005086891; WO2006015312; WO2006084272). Similarly, Linsley et al., (US 20030104426) and Radish et al., (US 20070154931) disclose gene markers genesets and methods of calculating gene expression template useful in classifying chronic myologenous leukemia patients.

In one embodiment, the method for identifying marker sets is as follows. After extraction and labeling of target polynucleotides, the expression of all markers (genes) in a sample X is compared to the expression of all markers in a standard or control. In one embodiment, the standard or control comprises target polynucleotide molecules derived from a sample from a cell sample not exposed to the Wee1 inhibitor. In another embodiment, the standard or control is a pool of target polynucleotide molecules. The pool may be derived from collected samples from a number of cancer individuals. In certain embodiments, the pool comprises samples taken from a number of individuals having cancers responsive to a Wee1 inhibitor. In another embodiment, the pool comprises an artificially-generated population of nucleic acids designed to approximate the level of nucleic acid derived from each marker found in a pool of marker-derived nucleic acids derived from tumor samples. In yet another embodiment, the pool is derived from cancer cell lines or cell line samples.

The comparison may be accomplished by any means known in the art. For example, expression levels of various markers may be assessed by separation of target polynucleotide molecules (e.g., RNA or cDNA) derived from the markers in agarose or polyacrylamide gels, followed by hybridization with marker-specific oligonucleotide probes. Alternatively, the comparison may be accomplished by the labeling of target polynucleotide molecules followed by separation on a sequencing gel. Polynucleotide samples are placed on the gel such that patient and control or standard polynucleotides are in adjacent lanes. Comparison of expression levels is accomplished visually or by means of densitometer. In one embodiment, the expression of all markers is assessed simultaneously by hybridization to a microarray. In each approach, markers meeting certain criteria are identified as associated with a cancer responsive to a Wee1 inhibitor.

Selection of a marker is based preferably on the difference in the expression level of at least one marker in a test sample compared to a standard or control sample. Selection may be made based upon a statistically significant up- or down regulation of the marker in the patient sample after treatment of Wee1 inhibitor. Selection may also be made by calculation of the statistical significance (i.e., the p-value) of the correlation between the expression of the marker and the condition or indication. Indeed, the larger the difference in expression of at least one biomarker detailed herein between the patient sample and the control or standard, the more informative the prediction. In certain embodiments, both selection criteria may be used. Thus, in one embodiment of the present invention, markers associated with a cancer responsive to a Wee 1 inhibitor are selected where the markers show both more than two-fold change (increase or decrease) in expression as compared to a standard, and the p-value for the correlation between the existence of cancerous condition and the change in marker expression is no more than 0.01 (i.e., is statistically significant).

The expression of the identified Wee1 responsive cancer markers may also be used to identify markers that can differentiate tumors into clinical types. In certain embodiments, beginning with a number of tumor samples, one may identify tumor specific markers by calculating the correlation coefficients between the clinical category or clinical parameter(s) and the linear, logarithmic or any transform of the expression ratio across all samples for each individual gene. Specifically, the correlation coefficient is calculated as:

ρ=(\vec{c}·\vec{r})/(∥\vec{c}∥·∥\vec{r}∥)
where \vec{c} represents the clinical parameters or categories and \vec{r} represents the linear, logarithmic or any transform of the ratio of expression between sample and control. Markers for which the coefficient of correlation exceeds a cutoff are identified as breast cancer-related markers specific for a particular clinical type. Such a cutoff or threshold corresponds to a certain significance of discriminating genes obtained by Monte Carlo simulations. The threshold depends upon the number of samples used; the threshold can be calculated as 3×1/\sqrt{n−3}, where 1/\sqrt{n−3} is the distribution width and n=the number of samples. In a specific embodiment, markers are chosen if the correlation coefficient is greater than about 0.3 or less than about −0.3.

Next, the significance of the correlation is calculated. This significance may be calculated by any statistical means by which such significance is calculated. In one example, a set of correlation data is generated using a Monte-Carlo technique to randomize the association between the expression difference of a particular marker and the clinical category. The frequency distribution of markers satisfying the criteria through calculation of correlation coefficients is compared to the number of markers satisfying the criteria in the data generated through the Monte-Carlo technique. The frequency distribution of markers satisfying the criteria in the Monte-Carlo runs is used to determine whether the number of markers selected by correlation with clinical data is significant.

Once a marker set is identified, the markers may be rank-ordered in order of significance of discrimination. One means of rank ordering is by the amplitude of correlation between the change in gene expression of the marker and the specific condition being discriminated. Another, preferred, means is to use a statistical metric. In one embodiment, the metric is a Fisher-like statistic:

$$t = \frac{( 〈 {x}_{1} 〉 - 〈 {x}_{2} 〉 )}{\sqrt{[ {σ}_{1}^{2} ( {n}_{1} - 1 ) + {σ}_{2}^{2} ( {n}_{2} - 1 ) ] / ( {n}_{1} + {n}_{2} - 1 ) / ( 1 / {n}_{1} + 1 / {n}_{2} )}}$$

In this equation, <x₁> is the error-weighted average of the log ratio of transcript expression measurements within a first diagnostic group (e.g., ER(−), <x₂> is the error-weighted average of log ratio within a second, related diagnostic group (e.g., ER(+)), σ₁ is the variance of the log ratio within the ER(−) group and n₁ is the number of samples for which valid measurements of log ratios are available, σ₂ is the variance of log ratio within the second diagnostic group (e.g., ER(+)), and n₂ is the number of samples for which valid measurements of log ratios are available. The t-value represents the variance-compensated difference between two means.

The rank-ordered marker set may be used to optimize the number of markers in the set used for discrimination. This is accomplished generally in a “leave one out” method as follows. In a first run, a subset, for example 5, of the markers from the top of the ranked list is used to generate a template, where out of X samples, X−1 are used to generate the template, and the status of the remaining sample is predicted. This process is repeated for every sample until every one of the X samples is predicted once. In a second run, additional markers, for example 5, are added, so that a template is now generated from 10 markers, and the outcome of the remaining sample is predicted. This process is repeated until the entire set of markers is used to generate the template. For each of the runs, type 1 error (false negative) and type 2 errors (false positive) are counted; the optimal number of markers is that number where the type 1 error rate, or type 2 error rate, or preferably the total of type 1 and type 2 error rate is lowest.

For prognostic markers, validation of the marker set may be accomplished by an additional statistic, a survival model. This statistic generates the probability of cancer as measured by, for example, tumor burden as a function of time since initial diagnosis. A number of models may be used, including Weibull, normal, log-normal, log logistic, log-exponential, or log-Rayleigh (Chapter 12 “Life Testing”, S-PLUS 2000 GUIDE TO STATISTICS, Vol. 2, p. 368 (2000)). For the “normal” model, the probability of distant metastases P at time t is calculated as

P=α×exp(−t²/τ²)
where α is fixed and equal to 1, and τ is a parameter to be fitted and measures the “expected lifetime”.

See U.S. Pat. No. 7,171,311 for each of the above referenced equations. The entire content of the above patent is incorporated by reference herein.

It will be apparent to those skilled in the art that the above methods, in particular the statistical methods, described above, are not limited to the identification of markers associated with a Wee1 inhibitor or Wee1 mediated cancer, but may be used to identify set of marker genes associated with any phenotype. The phenotype can be the presence or absence of a disease such as cancer, or the presence or absence of any identifying clinical condition associated with that cancer. In the disease context, the phenotype may be a prognosis such as a survival time, probability of distant metastases of a disease condition, or likelihood of a particular response to a therapeutic or prophylactic regimen. The phenotype need not be cancer, or a disease; the phenotype may be a nominal characteristic associated with a healthy individual.

In one embodiment, the similarity is represented by a correlation coefficient between the sample profile and the template. In another embodiment, a correlation coefficient above a correlation threshold indicates high similarity, whereas a correlation coefficient below the threshold indicates low similarity. In some embodiments, the correlation threshold is set as 0.3, 0.4, 0.5 or 0.6. In another embodiment, similarity between a sample profile and a template is represented by a distance between the sample profile and the template. In one embodiment, a distance below a given value indicates high similarity, whereas a distance equal to or greater than the given value indicates low similarity.

### Determination of Marker Gene Expression Levels

### A. Methods

The expression levels of the marker genes in a sample may be determined by any means known in the art. The expression level may be determined by isolating and determining the level (i.e., amount) of nucleic acid transcribed from each marker gene. Alternatively, or additionally, the level of specific proteins encoded by a marker gene may be determined.

The level of expression of specific marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label.

These examples are not intended to be limiting; other methods of determining RNA abundance are known in the art.

Finally, expression of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

### B. Microarrays

In some embodiments, polynucleotide microarrays are used to measure expression so that the expression status of each of the markers in one or more of the inventive gene sets, described herein, is assessed simultaneously. The microarrays of the invention preferably comprise at least 2, 3, 4, 5 or more of markers, or all of the markers, or any combination of markers, identified as classification-informative within a subject subset. The actual number of informative markers the microarray comprises will vary depending upon the particular condition of interest, the number of markers identified, and, optionally, the number of informative markers found to result in the least Type I error, Type II error, or Type I and Type II error in determination of an endpoint phenotype. As used herein, “Type I error” means a false positive and “Type II error” means a false negative; in the example of predicting a patient's therapeutic response to exposure to a Wee1 inhibitor, Type I error is the mischaracterization of an individual with a therapeutic response to a Wee1 inhibitor as being a non-responsive to Wee1 inhibitor treatment, and Type II error is the mischaracterization of an individual with no response to Wee1 inhibitor treatment as having a therapeutic response.

In specific embodiments, the invention provides polynucleotide arrays in which the markers identified for a particular subject subset comprise at least 50%, 60%, 70%, 80%, 85%, 90%, 95% or 98% of the probes on said array. In another specific embodiment, the microarray comprises a plurality of probes, wherein said plurality of probes comprise probes complementary and hybridizable to at least 75% of the Wee1 inhibitor exposure/prediction-informative markers identified for a particular patient subset. Microarrays of the invention, of course, may comprise probes complementary to and which are capable of hybridizing to Wee1 inhibitor prediction/evaluation-informative markers for a plurality of the subject subsets, or for each subject subset, identified for a particular condition. In furtherance thereof, a microarray of the invention comprises a plurality of probes complementary to and which hybridize to at least 75% of the Wee1 inhibitor prediction/evaluation-informative markers identified for each subject subset identified for the condition of interest, and wherein said probes, in total, are at least 50% of the probes on said microarray.

In yet another specific embodiment, the microarray is a commercially-available cDNA microarray that comprises at least two markers identified by the methods described herein. Preferably, a commercially-available cDNA microarray comprises all of the markers identified by the methods described herein as being informative for a patient subset for a particular condition. However, such a microarray may comprise at least 1, 2, 3, 4 or 5 of such markers, up to the maximum number of markers identified.

Any of the microarrays described herein may be provided in a sealed container in a kit.

In other embodiments, the array comprises a plurality of probes derived from markers listed in any of Tables 1 in combination with a plurality of other probes, derived from markers not listed in any of Tables 1, that are identified as informative for the prediction of sensitivity to a Wee1 inhibitor, evaluation of therapeutic response, etc.

### C. Polynucleotides Used to Measure the Products of the Biomarkers of the Invention

Polynucleotides capable of specifically or selectively binding to the mRNA transcripts encoding the polypeptide biomarkers of the invention are also contemplated. For example: oligonucleotides, cDNA, DNA, RNA, PCR products, synthetic DNA, synthetic RNA, or other combinations of naturally occurring or modified nucleotides which specifically and/or selectively hybridize to one or more of the RNA products of the biomarker of the invention are useful in accordance with the invention.

In a preferred embodiment, the oligonucleotides, cDNA, DNA, RNA, PCR products, synthetic DNA, synthetic RNA, or other combinations of naturally occurring or modified nucleotides oligonucleotides which both specifically and selectively hybridize to one or more of the RNA products of the biomarker of the invention are used.

To determine the (increased or decreased) expression levels of genes in the practice of the present invention, any method known in the art may be utilized. In one embodiment of the invention, expression based on detection of RNA which hybridizes to the genes identified and disclosed herein is used. This is readily performed by any RNA detection or amplification methods known or recognized as equivalent in the art such as, but not limited to, reverse transcription-PCR, and methods to detect the presence, or absence, of RNA stabilizing or destabilizing sequences.

Alternatively, expression based on detection of DNA status may be used. Detection of the DNA of an identified gene as may be used for genes that have increased expression in correlation with a particular outcome. This may be readily performed by PCR based methods known in the art, including, but not limited to, Q-PCR. Conversely, detection of the DNA of an identified gene as amplified may be used for genes that have increased expression in correlation with a particular treatment outcome. This may be readily performed by PCR based, fluorescent in situ hybridization (FISH) and chromosome in situ hybridization (CISH) methods known in the art.

### D. Techniques to Measure the RNA Products of the Biomarkers of the Invention

### 1) Real-Time PCR

In practice, a gene expression-based expression assay based on a small number of genes, i.e., about 1 to 3000 genes can be performed with relatively little effort using existing quantitative real-time PCR technology familiar to clinical laboratories. Quantitative real-time PCR measures PCR product accumulation through a dual-labeled fluorigenic probe. A variety of normalization methods may be used, such as an internal competitor for each target sequence, a normalization gene contained within the sample, or a housekeeping gene. Sufficient RNA for real time PCR can be isolated from low milligram quantities from a subject. Quantitative thermal cyclers may now be used with microfluidics cards preloaded with reagents making routine clinical use of multigene expression-based assays a realistic goal.

The gene markers of the various inventive genesets or a subset of genes selected from the inventive genesets, which are assayed according to the present invention are typically in the form of total RNA or mRNA or reverse transcribed total RNA or mRNA. General methods for total and mRNA extraction are well known in the art and are disclosed in standard textbooks of molecular biology, including Ausubel et al., Current Protocols of Molecular Biology, John Wiley and Sons (1997). RNA isolation can also be performed using purification kit, buffer set and protease from commercial manufacturers, such as Qiagen (Valencia, Calif.) and Ambion (Austin, Tex.), according to the manufacturer's instructions.

TAQman quantitative real-time PCR can be performed using commercially available PCR reagents (Applied Biosystems, Foster City, Calif.) and equipment, such as ABI Prism 7900HT Sequence Detection System (Applied Biosystems) according the manufacturer's instructions. The system consists of a thermocycler, laser, charge-coupled device (CCD), camera, and computer. The system amplifies samples in a 96-well or 384-well format on a thermocycler. During amplification, laser-induced fluorescent signal is collected in real-time through fiber-optics cables for all 96 wells, and detected at the CCD. The system includes software for running the instrument and for analyzing the data.

Based upon the marker gene sets identified in the present invention, a real-time PCR TAQman assay can be used to make gene expression measurements and perform the classification methods described herein. As is apparent to a person of skill in the art, a wide variety of oligonucleotide primers and probes that are complementary to or hybridize to the markers of the invention may be selected based upon the marker transcript sequences set forth in the Sequence Listing.

### 2) Array Hybridization

The polynucleotide used to measure the RNA products of the invention can be used as nucleic acid members stably associated with a support to comprise an array according to one aspect of the invention. The length of a nucleic acid member can range from 8 to 1000 nucleotides in length and are chosen so as to be specific for the RNA products of the biomarkers of the invention. In one embodiment, these members are selective for the RNA products of the invention. The nucleic acid members may be single or double stranded, and/or may be oligonucleotides or PCR fragments amplified from cDNA. Preferably oligonucleotides are approximately 20-30 nucleotides in length. ESTs are preferably 100 to 600 nucleotides in length. It will be understood to a person skilled in the art that one can utilize portions of the expressed regions of the biomarkers of the invention as a probe on the array. More particularly oligonucleotides complementary to the genes of the invention and cDNA or ESTs derived from the genes of the invention are useful. For oligonucleotide based arrays, the selection of oligonucleotides corresponding to the gene of interest which are useful as probes is well understood in the art. More particularly it is important to choose regions which will permit hybridization to the target nucleic acids. Factors such as the Tm of the oligonucleotide, the percent GC content, the degree of secondary structure and the length of nucleic acid are important factors. See for example U.S. Pat. No. 6,551,784.

### 3) Construction of a Nucleic Acid Array

In the proposed methods, an array of nucleic acid members stably associated with the surface of a substantially support is contacted with a sample comprising target nucleic acids under hybridization conditions sufficient to produce a hybridization pattern of complementary nucleic acid members/target complexes in which one or more complementary nucleic acid members at unique positions on the array specifically hybridize to target nucleic acids. The identity of target nucleic acids which hybridize can be determined with reference to location of nucleic acid members on the array.

The nucleic acid members may be produced using established techniques such as polymerase chain reaction (PCR) and reverse transcription (RT). These methods are similar to those currently known in the art (see e.g., PCR Strategies, Michael A. Innis (Editor), et al. (1995) and PCR: Introduction to Biotechniques Series, C. R. Newton, A. Graham (1997)). Amplified nucleic acids are purified by methods well known in the art (e.g., column purification or alcohol precipitation). A nucleic acid is considered pure when it has been isolated so as to be substantially free of primers and incomplete products produced during the synthesis of the desired nucleic acid. Preferably, a purified nucleic acid will also be substantially free of contaminants which may hinder or otherwise mask the specific binding activity of the molecule.

An array, according to one aspect of the invention, comprises a plurality of nucleic acids attached to one surface of a support at a density exceeding 20 different nucleic acids/cm², wherein each of the nucleic acids is attached to the surface of the support in a non-identical pre-selected region (e.g. a microarray). Each associated sample on the array comprises a nucleic acid composition, of known identity, usually of known sequence, as described in greater detail below. Any conceivable substrate may be employed in the invention.

In one embodiment, the nucleic acid attached to the surface of the support is DNA. In one embodiment, the nucleic acid attached to the surface of the support is cDNA or RNA. In another embodiment, the nucleic acid attached to the surface of the support is cDNA synthesized by polymerase chain reaction (PCR). Usually, a nucleic acid member in the array, according to the invention, is at least 10, 25, 50, 60 nucleotides in length. In one embodiment, a nucleic acid member is at least 150 nucleotides in length. Preferably, a nucleic acid member is less than 1000 nucleotides in length. More preferably, a nucleic acid member is less than 500 nucleotides in length.

In the arrays of the invention, the nucleic acid compositions are stably associated with the surface of a support, where the support may be a flexible or rigid support. By “stably associated” is meant that each nucleic acid member maintains a unique position relative to the support under hybridization and washing conditions. As such, the samples are non-covalently or covalently stably associated with the support surface. Examples of non-covalent association include non-specific adsorption, binding based on electrostatic interactions (e.g., ion pair interactions), hydrophobic interactions, hydrogen bonding interactions, specific binding through a specific binding pair member covalently attached to the support surface, and the like. Examples of covalent binding include covalent bonds formed between the nucleic acids and a functional group present on the surface of the rigid support (e.g., —OH), where the functional group may be naturally occurring or present as a member of an introduced linking group, as described in greater detail below.

The amount of nucleic acid present in each composition will be sufficient to provide for adequate hybridization and detection of target nucleic acid sequences during the assay in which the array is employed. Generally, the amount of each nucleic acid member stably associated with the support of the array is at least about 0.001 ng, preferably at least about 0.02 ng and more preferably at least about 0.05 ng, where the amount may be as high as 1000 ng or higher, but will usually not exceed about 20 ng. Where the nucleic acid member is “spotted” onto the support in a spot comprising an overall circular dimension, the diameter of the “spot” will generally range from about 10 to 5,000 μm, usually from about 20 to 2,000 μm and more usually from about 100 to 200 μm.

Control nucleic acid members may be present on the array including nucleic acid members comprising oligonucleotides or nucleic acids corresponding to genomic DNA, housekeeping genes, vector sequences, plant nucleic acid sequence, negative and positive control genes, and the like. Control nucleic acid members are calibrating or control genes whose function is not to tell whether a particular “key” gene of interest is expressed, but rather to provide other useful information, such as background or basal level of expression.

Other control nucleic acids are spotted on the array and used as target expression control nucleic acids and mismatch control nucleotides to monitor non-specific binding or cross-hybridization to a nucleic acid in the sample other than the target to which the probe is directed. Mismatch probes thus indicate whether a hybridization is specific or not. For example, if the target is present, the perfectly matched probes should be consistently brighter than the mismatched probes. In addition, if all control mismatches are present, the mismatch probes are used to detect a mutation.

Numerous methods may be used for attachment of the nucleic acid members of the invention to the substrate (a process referred to as “spotting”). For example, nucleic acids are attached using the techniques of, for example U.S. Pat. No. 5,807,522, which is incorporated herein by reference for teaching methods of polymer attachment. Alternatively, spotting may be carried out using contact printing technology as is known in the art.

The measuring of the expression of the RNA product of the invention can be done by using those polynucleotides which are specific and/or selective for the RNA products of the invention to quantitate the expression of the RNA product. In a specific embodiment of the invention, the polynucleotides which are specific and/or selective for the RNA products are probes or primers. In one embodiment, these polynucleotides are in the form of nucleic acid probes which can be spotted onto an array to measure RNA from the sample of an individual to be measured. In another embodiment, commercial arrays can be used to measure the expression of the RNA product. In yet another embodiment, the polynucleotides which are specific and/or selective for the RNA products of the invention are used in the form of probes and primers in techniques such as quantitative real-time RT PCR, using for example SYBR®Green, or using TaqMan® or Molecular Beacon techniques, where the polynucleotides used are used in the form of a forward primer, a reverse primer, a TaqMan labeled probe or a Molecular Beacon labeled probe.

In embodiments where only one or a two genes are to be analyzed, the nucleic acid derived from the sample cell(s) may be preferentially amplified by use of appropriate primers such that only the genes to be analyzed are amplified to reduce background signals from other genes expressed in the breast cell. Alternatively, and where multiple genes are to be analyzed or where very few cells (or one cell) is used, the nucleic acid from the sample may be globally amplified before hybridization to the immobilized polynucleotides. Of course RNA, or the cDNA counterpart thereof may be directly labeled and used, without amplification, by methods known in the art.

4) Use of a Microarray

A “microarray” is a linear or two-dimensional array of preferably discrete regions, each having a defined area, formed on the surface of a solid support such as, but not limited to, glass, plastic, or synthetic membrane. The density of the discrete regions on a microarray is determined by the total numbers of immobilized polynucleotides to be detected on the surface of a single solid phase support, preferably at least about 50/cm², more preferably at least about 100/cm², even more preferably at least about 500/cm², but preferably below about 1,000/cm². Preferably, the arrays contain less than about 500, about 1000, about 1500, about 2000, about 2500, or about 3000 immobilized polynucleotides in total. As used herein, a DNA microarray is an array of oligonucleotides or polynucleotides placed on a chip or other surfaces used to hybridize to amplified or cloned polynucleotides from a sample. Since the position of each particular group of primers in the array is known, the identities of a sample polynucleotides can be determined based on their binding to a particular position in the microarray.

Determining gene expression levels may be accomplished utilizing microarrays. Generally, the following steps may be involved: (a) obtaining an mRNA sample from a subject and preparing labeled nucleic acids therefrom (the “target nucleic acids” or “targets”); (b) contacting the target nucleic acids with an array under conditions sufficient for the target nucleic acids to bind to the corresponding probes on the array, for example, by hybridization or specific binding; (c) optional removal of unbound targets from the array; (d) detecting the bound targets, and (e) analyzing the results, for example, using computer based analysis methods. As used herein, “nucleic acid probes” or “probes” are nucleic acids attached to the array, whereas “target nucleic acids” are nucleic acids that are hybridized to the array.

Nucleic acid specimens may be obtained from a subject to be tested using either “invasive” or “non-invasive” sampling means. A sampling means is said to be “invasive” if it involves the collection of nucleic acids from within the skin or organs of an animal (including murine, human, ovine, equine, bovine, porcine, canine, or feline animal). Examples of invasive methods include, for example, blood collection, semen collection, needle biopsy, pleural aspiration, umbilical cord biopsy. Examples of such methods are discussed by Kim, et al., (J. Virol. 66:3879-3882, 1992); Biswas, et al., (Ann. NY Acad. Sci. 590:582-583, 1990); and Biswas, et al., (J. Clin. Microbiol. 29:2228-2233, 1991).

In contrast, a “non-invasive” sampling means is one in which the nucleic acid molecules are recovered from an internal or external surface of the animal. Examples of such “non-invasive” sampling means include, for example, “swabbing,” collection of tears, saliva, urine, fecal material etc.

In one embodiment of the present invention, one or more cells from the subject to be tested are obtained and RNA is isolated from the cells. In one embodiment, a sample of cells is obtained from the subject. It is also possible to obtain a cell sample from a subject, and then to enrich the sample for a desired cell type. For example, cells may be isolated from other cells using a variety of techniques, such as isolation with an antibody binding to an epitope on the cell surface of the desired cell type. Where the desired cells are in a solid tissue, particular cells may be dissected, for example, by microdissection or by laser capture microdissection (LCM) (see, e.g., Bonner, et al., Science 278:1481, 1997; Emmert-Buck, et al., Science 274:998, 1996; Fend, et al., Am. J. Path. 154:61, 1999; and Murakami, et al., Kidney hit. 58:1346, 2000).

RNA may be extracted from tissue or cell samples by a variety of methods, for example, guanidium thiocyanate lysis followed by CsCl centrifugation (Chirgwin, et al., Biochemistry 18:5294-5299, 1979). RNA from single cells may be obtained as described in methods for preparing cDNA libraries from single cells (see, e.g., Dulac, Curr. Top. Dev. Biol. 36:245, 1998; Jena, et al., J. Immunol. Methods 190:199, 1996).

The RNA sample can be further enriched for a particular species. In one embodiment, for example, poly(A)+RNA may be isolated from an RNA sample. In another embodiment, the RNA population may be enriched for sequences of interest by primer-specific cDNA synthesis, or multiple rounds of linear amplification based on cDNA synthesis and template-directed in vitro transcription (see, e.g., Wang, et al., Proc. Natl. Acad. Sci. USA 86:9717, 1989; Dulac, et al., supra; Jena, et al., supra). In addition, the population of RNA, enriched or not in particular species or sequences, may be further amplified by a variety of amplification methods including, for example, PCR; ligase chain reaction (LCR) (see, e.g., Wu and Wallace, Genomics 4:560, 1989; Landegren, et al., Science 241:1077, 1988); self-sustained sequence replication (SSR) (see, e.g., Guatelli, et al., Proc. Natl. Acad. Sci. USA 87:1874, 1990); nucleic acid based sequence amplification (NASBA) and transcription amplification (see, e.g., Kwoh, et al., Proc. Natl. Acad. Sci. USA 86:1173, 1989). Methods for PCR technology are well known in the art (see, e.g., PCR Technology: Principles and Applications for DNA Amplification (ed. H. A. Erlich, Freeman Press, N.Y., N.Y., 1992); PCR Protocols: A Guide to Methods and Applications (eds. Innis, et al., Academic Press, San Diego, Calif., 1990); Manila, et al., Nucleic Acids Res. 19:4967, 1991; Eckert, et al., PCR Methods and Applications 1:17, 1991; PCR (eds. McPherson et al., IRL Press, Oxford); and U.S. Pat. No. 4,683,202). Methods of amplification are described, for example, by Ohyama, et al., (BioTechniques 29:530, 2000); Luo, et al., (Nat. Med. 5:117, 1999); Hegde, et al., (BioTechniques 29:548, 2000); Kacharmina, et al., (Meth. Enzymol. 303:3, 1999); Livesey, et al., Curr. Biol. 10:301, 2000); Spirin, et al., (Invest. Ophtalmol. Vis. Sci. 40:3108, 1999); and Sakai, et al., (Anal. Biochem. 287:32, 2000). RNA amplification and cDNA synthesis may also be conducted in cells in situ (see, e.g., Eberwine, et al. Proc. Natl. Acad. Sci. USA 89:3010, 1992).

In yet another embodiment of the invention, all or part of a disclosed marker sequence may be amplified and detected by methods such as the polymerase chain reaction (PCR) and variations thereof, such as, but not limited to, quantitative PCR (Q-PCR), reverse transcription PCR (RT-PCR), and real-time PCR, optionally real-time RT-PCR. Such methods would utilize one or two primers that are complementary to portions of a disclosed sequence, where the primers are used to prime nucleic acid synthesis.

The newly synthesized nucleic acids are optionally labeled and may be detected directly or by hybridization to a polynucleotide of the invention.

The nucleic acid molecules may be labeled to permit detection of hybridization of the nucleic acid molecules to a microarray. That is, the probe may comprise a member of a signal producing system and thus, is detectable, either directly or through combined action with one or more additional members of a signal producing system. For example, the nucleic acids may be labeled with a fluorescently labeled dNTP (see, e.g., Kricka, 1992, Nonisotopic DNA Probe Techniques, Academic Press San Diego, Calif.), biotinylated dNTPs or rNTP followed by addition of labeled streptavidin, chemiluminescent labels, or isotopes. Another example of labels include “molecular beacons” as described in Tyagi and Kramer (Nature Biotech. 14:303, 1996). The newly synthesized nucleic acids may be contacted with polynucleotides (containing sequences) of the invention under conditions which allow for their hybridization. Hybridization may be also determined, for example, by plasmon resonance (see, e.g., Thiel, et al. Anal. Chem. 69:4948, 1997).

In one embodiment, a plurality e.g., 2 sets of target nucleic acids are labeled and used in one hybridization reaction (“multiplex” analysis). For example, one set of nucleic acids may correspond to RNA from one cell and another set of nucleic acids may correspond to RNA from another cell. The plurality of sets of nucleic acids may be labeled with different labels, for example, different fluorescent labels (e.g., fluorescein and rhodamine) which have distinct emission spectra so that they can be distinguished. The sets may then be mixed and hybridized simultaneously to one microarray (see, e.g., Shena, et al., Science 270:467-470, 1995).

A number of different microarray configurations and methods for their production are known to those of skill in the art and are disclosed in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 6,022,963; 6,077,674; and 6,156,501; Shena, et al., Tibtech 16:301, 1998; Duggan, et al., Nat. Genet. 21:10, 1999; Bowtell, et al., Nat. Genet. 21:25, 1999; Lipshutz, et al., 21 Nature Genet. 20-24, 1999; Blanchard, et al., 11 Biosensors and Bioelectronics, 687-90, 1996; Maskos, et al., 21 Nucleic Acids Res. 4663-69, 1993; Hughes, et al., Nat. Biotechol. (2001) 19:342; the disclosures of which are herein incorporated by reference. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219; the disclosures of which are herein incorporated by reference.

In one embodiment, an array of oligonucleotides may be synthesized on a solid support. Exemplary solid supports include glass, plastics, polymers, metals, metalloids, ceramics, organics, etc. Using chip masking technologies and photoprotective chemistry, it is possible to generate ordered arrays of nucleic acid probes. These arrays, which are known, for example, as “DNA chips” or very large scale immobilized polymer arrays (“VLSIPS®” arrays), may include millions of defined probe regions on a substrate having an area of about 1 cm² to several cm², thereby incorporating from a few to millions of probes (see, e.g., U.S. Pat. No. 5,631,734).

To compare expression levels, labeled nucleic acids may be contacted with the array under conditions sufficient for binding between the target nucleic acid and the probe on the array. In one embodiment, the hybridization conditions may be selected to provide for the desired level of hybridization specificity; that is, conditions sufficient for hybridization to occur between the labeled nucleic acids and probes on the microarray.

Hybridization may be carried out in conditions permitting essentially specific hybridization. The length and GC content of the nucleic acid will determine the thermal melting point and thus, the hybridization conditions necessary for obtaining specific hybridization of the probe to the target nucleic acid. These factors are well known to a person of skill in the art, and may also be tested in assays. An extensive guide to nucleic acid hybridization may be found in Tijssen, et al. (Laboratory Techniques in Biochemistry and Molecular Biology, Vol. 24: Hybridization With Nucleic Acid Probes, P. Tijssen, ed. Elsevier, N.Y., (1993)).

The methods described above will result in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

One such method of detection utilizes an array scanner that is commercially available (Affymetrix, Santa Clara, Calif.), for example, the 417® Arrayer, the 418® Array Scanner, or the Agilent GeneArray® Scanner. This scanner is controlled from a system computer with an interface and easy-to-use software tools. The output may be directly imported into or directly read by a variety of software applications. Exemplary scanning devices are described in, for example, U.S. Pat. Nos. 5,143,854 and 5,424,186.

### 5) Administration of Wee1 Inhibitors

Cancers amenable to treatment with a Wee1 inhibitor include but are not limited to acute myelogenous leukemia (AML), chronic myelogenous leukemia (CML), acute lymphocytic leukemia (ALL), and chronic lymphocytic leukemia, Kaposi's sarcoma; breast cancers; bone cancers, brain cancers, cancers of the head and neck, gallbladder and bile duct cancers, cancers of the retina, cancers of the esophagus, gastric cancers, multiple myeloma, ovarian cancer, uterine cancer, thyroid cancer, testicular cancer, endometrial cancer, melanoma, colorectal cancer, bladder cancer, prostate cancer, lung cancer, pancreatic cancer, sarcomas, Wilms' tumor, cervical cancer, skin cancers, nasopharyngeal carcinoma, liposarcoma, epithelial carcinoma, renal cell carcinoma, gallbladder adenocarcinoma, parotid adenocarcinoma, and endometrial sarcoma.

The Wee1 inhibitor can be administered by any known administration method known to a person skilled in the art. Examples of routes of administration include but are not limited to oral, parenteral, intraperitoneal, intravenous, intraarterial, transdermal, sublingual, intramuscular, rectal, transbuccal, intranasal, liposomal, via inhalation, vaginal, intraoccular, via local delivery by catheter or stent, subcutaneous, intraadiposal, intraarticular, intrathecal, or in a slow release dosage form.

The Wee1 inhibitors or a pharmaceutically acceptable salt or hydrate thereof, can be administered in accordance with any dose and dosing schedule that, achieves a dose effective to treat cancer. For example, Wee1 inhibitors can be administered in a total daily dose of up to 1000 mg, preferably orally, once, twice or three times daily, continuously (every day) or intermittently (e.g., 3-5 days a week).

A Wee1 inhibitor may also be administered in combination with an anti-cancer agent, wherein the amount of Wee1 and the amount of the anti-cancer agent together comprise a therapeutically effective amount. The combination therapy can provide a therapeutic advantage in view of the differential toxicity associated with the two treatment modalities. For example, treatment with Wee1 inhibitors can lead to a particular toxicity that is not seen with the anti-cancer agent, and vice versa. As such, this differential toxicity can permit each treatment to be administered at a dose at which said toxicities do not exist or are minimal, such that together the combination therapy provides a therapeutic dose while avoiding the toxicities of each of the constituents of the combination agents. Furthermore, when the therapeutic effects achieved as a result of the combination treatment are enhanced or synergistic, for example, significantly better than additive therapeutic effects, the doses of each of the agents can be reduced even further, thus lowering the associated toxicities to an even greater extent.

Wee1 inhibitor can be combined with chemotherapy and radiotherapy. Wee1 inhibitor is also combined with an anti-cancer agent, but is preferably combined with a DNA damaging agents. Examples of such anti-cancer agent used in a combination treatment with Wee1 inhibitors are for example, but not limited to, gemcitabine, cisplatin, carboplatin, 5-fluorouracil, pemetrexed, doxorubicin, camptothecin and mitomycin.

In one embodiment, a Wee1 inhibitor is administered in a pharmaceutical composition, preferably suitable for oral administration. In another embodiment, Wee1 is administered orally in a gelating capsule, which can comprise excipients such as microcrystalline cellulose, croscarmellose sodium and magnesium stearate.

The Wee1 inhibitors can be administered in a total daily dose that may vary from patient to patient, and may be administered at varying dosage schedules. Suitable dosages are total daily dosage of between about 25-4000 mg/m² administered orally once-daily, twice-daily or three times-daily, continuous (every day) or intermittently (e.g. 3-5 days a week). Furthermore, the compositions may be administered in cycles, with rest periods in between the cycles (e.g. treatment for two to eight weeks with a rest period of up to a week between treatments).

Other treatment combinations and dosing regiments are set forth in WO 2007/126122, WO2007/126128 and WO 2008/133866.

It is apparent to a person skilled in the art that any one or more of the specific dosages and dosage schedules of the Wee1 inhibitors, is also applicable to any one or more of the anti-cancer agents to be used in the combination treatment. Moreover, the specific dosage and dosage schedule of the anti-cancer agent can further vary, and the optimal dose, dosing schedule and route of administration will be determined based upon the specific anti-cancer agent that is being used.

### 6) Materials and Methods

### 1. Test Compound

1) Compound A (Wee 1 Inhibitor)
   Compound A 2-allyl-1-[6-(1-hydroxy-1-methylethyl)pyridin-2-yl]-6-{[4-(4-methylpiperazin-1-yl)phenyl]amino}-1,2-dihydro-3H-pyrazolo[3,4-d]pyrimidin-3-one is disclosed as Example 53 in WO2007/126128. Compound A was synthesized according to the description of WO2007/126128, and was stored at −20. Purity was 99.3%. Compound A was dissolved in dimethyl sulphoxide (DMSO) (SIGMA, #D2650).
2) Gemcitabine (Gem): Gemzar® Injection (Eli Lilly Japan K.K.) was dissolved in Phosphate Buffered Saline, pH 7.4 (PBS) (Invitrogen, #10010-049) and stored at −20° C.
3) Carboplatin: Carboplatin (SIGMA, #C2538) was dissolved in PBS and stored at −20° C.
4) Cisplatin: Cisplatin (SIGMA, #P4394) was dissolved in PBS and stored at −20° C.

### 2. Cell Lines and Culture Media

22 NSCLC cell lines with deficient p53 status, NCI-H520, NCI-H661, NCI-H1838, NCI-H2135, NCI-H2444, PC-13, NCI-H1915, NCI-H1975, UCM-11, NCI-H1299, NCI-H727, NCI-H358, NCI-H1568, NCI-H2030, NCI-H1437, SK-MES-1, NCI-H1993, NCI-H2122, NCI-H1703, NCI-H2023, NCI-H2172, NCI-H441, obtained from American Type Culture Collection, and were cultured according to the supplier's instructions.

### 3. Cytotoxicity Assay

Each cell line was cultured for 24 hours, and then DNA damaging agent (Gemcitabine at 1˜300 nM, Carboplatin at 1˜300 uM or Cisplatin at 0.1˜30 uM) were added and continued to be cultured for another 24 hours. Wed inhibitor (Compound A) was added to the cultured cells at 30, 100 and 300 nM, and they were incubated for additional 24 hours. Then, cell viability was measured with a WST-8 kit (KISHIDA CHEMICAL CO., LTD) using a SpectraMax Plus384 plate reader (Molecular Devices Corporation). The sensitivity to the combination therapy was shown as Bliss additivism index.

### 4. Microarray Analysis

The 22 NSCLC cell lines with deficient p53 status were analyzed for the cytotoxicity assay. Among the 22 cell lines, top 10 higher-sensitive cell lines and bottom 10 lower-sensitive cell lines were classified as hyper-responders and normal-responders respectively. The genes which were differentially expressed between the hyper-responder and normal-responder cancer cell lines were extracted by applying ANOVA test (P<=0.01). The genes whose Pearson correlation between Bliss additivism index and gene expression level was statistically significant (P<=0.01) were further selected down. The P value of hypergeometric test represents the chance of seeing the observed number of overlap genes (or more) between the input set and the comparison set if input genes are randomly selected. Leave-one-out cross-validation (LOOCV) involves using a single Bliss index of one cell line from the original sample containing the 20 cell lines as the validation data, and the remaining 19 data as the training data. This is repeated such that each observation in the sample is used once as the validation data.

### EXAMPLES

### 1. Determination of Sensitivity of 22 Non Small Cell Lung Cancers to the Combination of Wee1 Inhibitor and DNA Damaging Combination

The 22 NSCLC cell lines with deficient p53 status were treated with Compound A/Gemcitabine, Compound A/Carboplatin and Compound A/Cisplatin respectively as shown in Materials and Methods, supra.

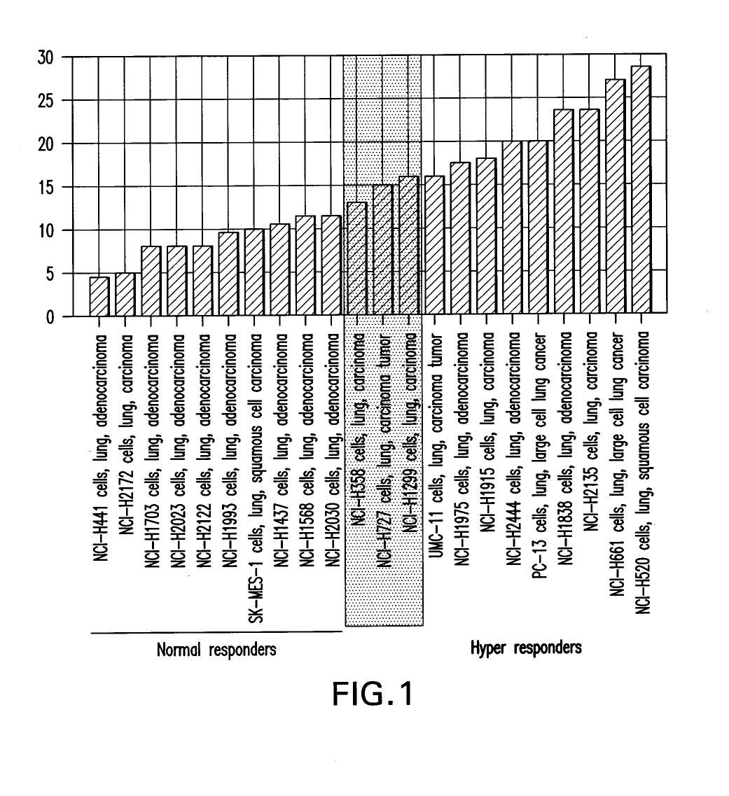

The viability of the cell lines was measured by a cytotoxic assay, and sensitivity of the cells to the combination therapy was shown as bliss additivism index. The result of the treatment with Compound A/Cisplatin is shown in FIG. 1. As shown in FIG. 1, the synergistic effect (excess over Bliss additivism) was variable, ranging from 4 to 32 among the cell lines treated with the Compound A/Cisplatin combination. Among the 22 cell lines, top 10 high-sensitivity cell lines and bottom 10 low-sensitivity cell lines were classified as hyper-responders and normal-responders respectively.

Similar result e.g., variable sensitivity was observed for cell lines treated with each of the following combinations—Compound A/Gemcitabine and Compound A/Carboplatin. These results inferred other factors in addition to p53 status could affect the sensitivity to Wee1 inhibitor/DNA damaging combination.

### 2. Identification of Responder Signature Genes Differentially Expressed Between Hyper Responder and Normal Responders

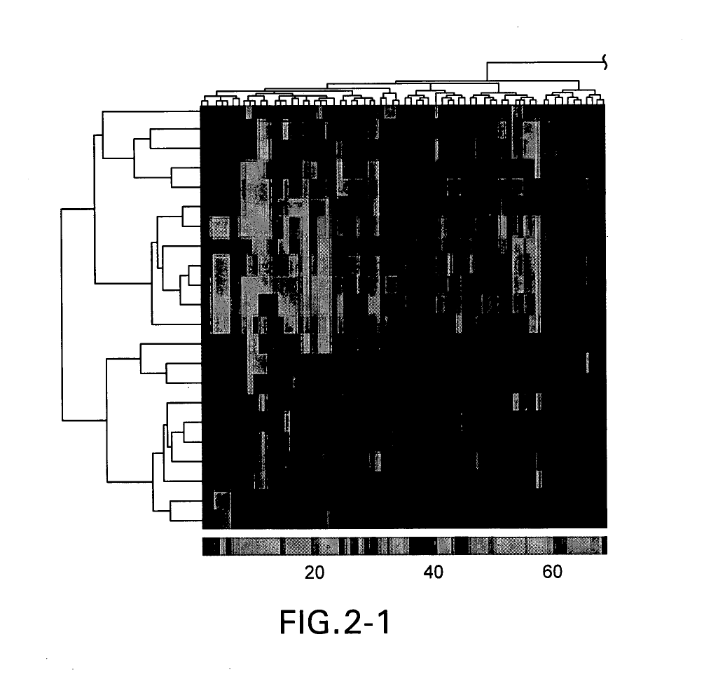

Genes differentially expressed between 10 hyper and 10 normal responder lung cancer cell lines to Compound A/Cisplatin combination treatment were selected. Genes whose expression patterns were highly correlated with the synergistic index (see materials and methods section supra) were reviewed with the objective of narrowing the gene set(s) for further study. As a result, 117 genes were selected as the signature genes for hyper-responder to the Compound A/Cisplatin treatment. The selected signature genes could correctly distinguish the two groups by hierarchical clustering analysis as shown in FIG. 2. The 117 genes are shown in Table 2 and Table 3.

TABLE 2
Down-regulated genes in hyper-responder cells

ALDH3A2
ALPK1
APOL2
ATP1B1
B4GALT1
BACE2
BANK1
BCL6
C14orf149
C21orf129
CCL18
CCNI
CEBPD
CLDN1
CLEC4D
CLINT1
CNGB3
CRTC3
CTSL1
CYP1B1
DACT2
EFNA1
FAM107B
FAM26B
GUSBP1
HRB
IL28B
JUNB
KCNK1
KIAA0494
LAPTM4A
LITAF
MAN1A2
MED18
MIDN
NCOA7
NR1H2
NT5C2
NUDT9
PDLIM5
PDXK
PMM2
RLBP1L1
S100P
SERTAD4
SHC1
SLC16A7
SLPI
SMAD2
TLK1
TMEM164
TMEM87B
TPRG1
YIPF6
ZFAND5

TABLE 3
Up-regulated genes in hyper-responder cells

ANKRD26
APLN
ASGR1
ATP4A
BCAT1
CCDC112
CCDC136
CKAP5
CKB
CLPP
CSRP2
DNAJC18
DUSP6
FAM118B
FAM130A1
FAM29A
FBXL12
FDX1L
FOXRED1
GADD45GIP1
GAL3ST4
GDF9
HMGN3
HPCAL4
HPS4
LIMD2
LOXL3
LSM7
MAD1L1
MAGED4B
MCM8
MEX3A
MRPL4
MYH3
MYLK2
NAT10
NELL2
NFU1
NXF1
OSBPL6
PANK2
PGF
PHF14
PIK3C2A
PLEKHO1
RRAGD
SAAL1
SALL2
SFMBT1
SNAPC3
SPOP
SPRY2
SSR3
TIMM44
TLK2
TTC9C
TUBB2A
TUBB2B
TUBB4
WDR8
XPO4
ZNF653

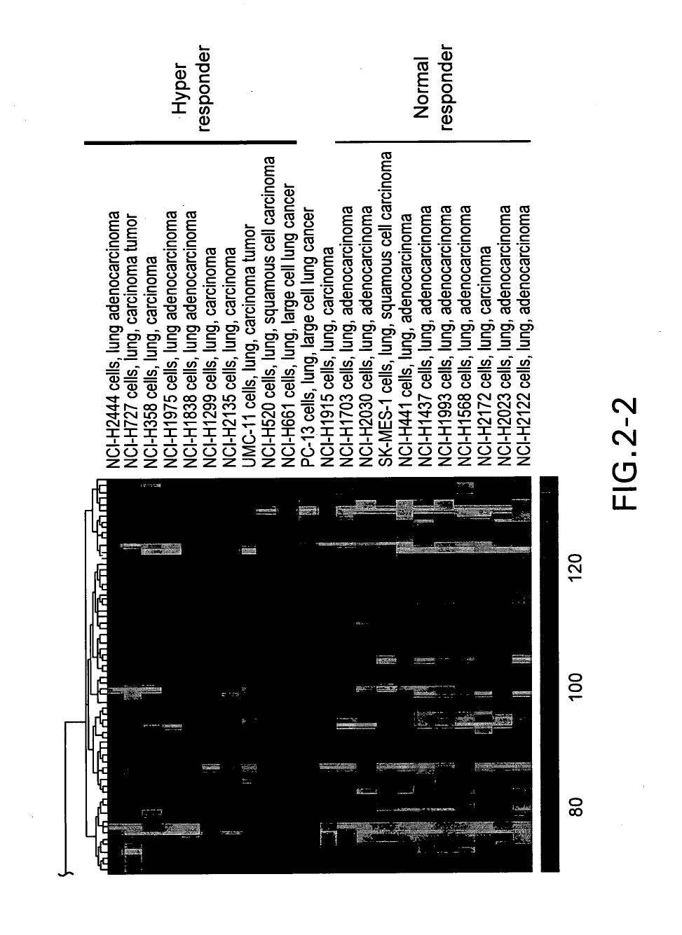

Leave-one-out-cross-validation test was carried out, and the results are shown in FIG. 3. The data shows that the prediction accuracy by this method using the signature genes is 80%.

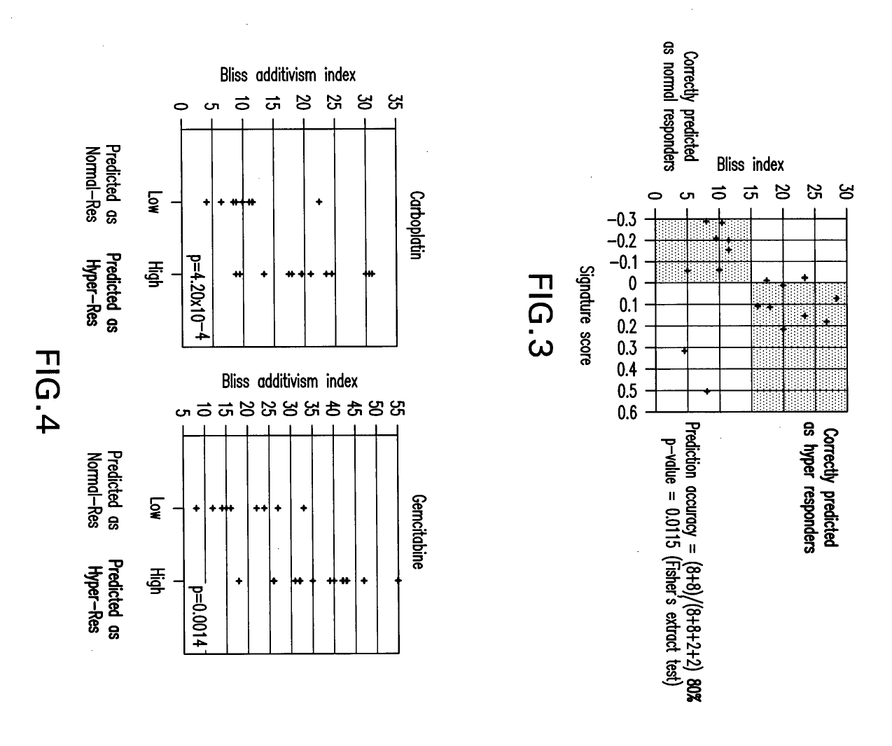

The test also examined whether the signature genes could be used to predict the sensitivities to one of Compound A/Carboplatin or Compound A/Gemcitabine combination treatment. As shown in FIG. 4, the data demonstrates that the 134 signature genes could distinguish hyper- and normal-responders for both Compound A/Carboplatin combination treatment (p value=4.2×10⁻⁴) and Compound A/Gemcitabine combination treatment (p value=1.4×10⁻³) respectively. These results indicate that the selected signature genes were useful in predicting hyper responders among p53-deficient cancers.

### 3. Expression Ratio of MAD1/SMAD2 Predicts Hyper- and Normal-Responder Cancers

Hypergeometric test was performed for the 134 signature genes, and it found that mitotic cell cycle regulated genes were significantly condensed in the gene set (Table 4: P=0.0079). This is in consistent with the previous report that mitotic cell cycle regulatory genes relate to the sensitivity to G2 checkpoint abrogators.

TABLE 4
Hypergeometric test for signature genes

| Biological Process | | | | | | |
|---|---|---|---|---|---|---|
| (GO) | P-value | Signature | Overlap | Set | Background | Gene |
| Regulation of cell | 0.00571 | 92 | 8 | 440 | 14941 | SPRY2; CCNI; JUNB; SMAD2; |
| cycle | | | | | | SHC1; MAD1L1; GADD45GIP1; |
| | | | | | | CKAP5 |
| Mitotic cell cycle | 0.00786 | 92 | 9 | 564 | 14941 | TUBB4; JUNB; SMAD2; |
| | | | | | | BCAT1; SHC1; MAD1L1; |
| | | | | | | MCM8; GADD45GIP1; CKAP5 |
| Cell cycle phase | 0.00821 | 92 | 9 | 568 | 14941 | TUBB4; TLK2; SMAD2; BCAT1; |
| | | | | | | SHC1; MAD1L1; MCM8; |
| | | | | | | GADD45GIP1; CKAP5 |

In order to find use in clinical trial, e.g., stratifying patient population etc. the inventors further narrowed the signature gene set(s) and identified two cell cycle related genes, annotated in the hypergeometric test. Theses two genes are designated herein as MAD1L1 (MAD1) and SMAD2.

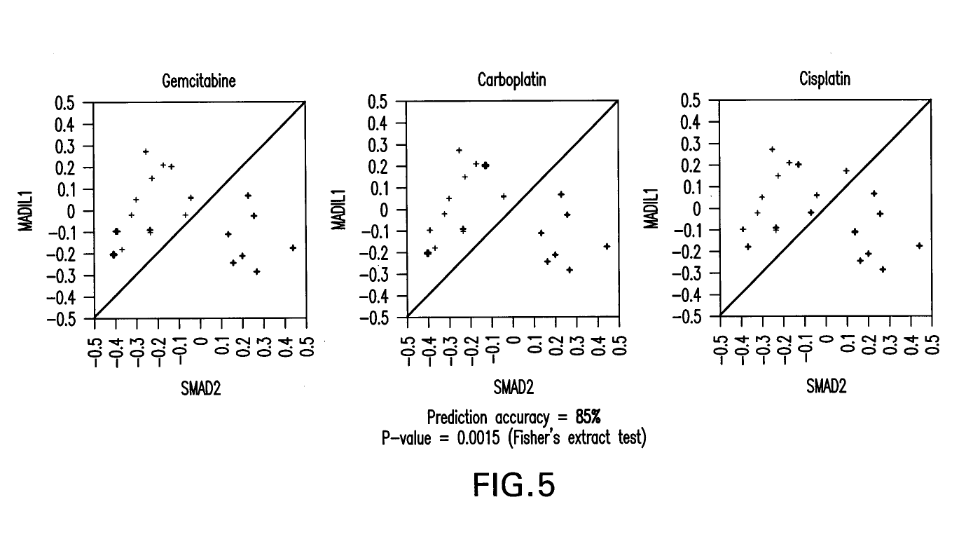

The inventors next examined whether the selected two genes could be used to identify a hyper responder and predict its sensitivity to a Wee1 inhibitor. As shown in FIG. 5, the expression ratio of MAD1 to SMAD2 was useful in being able to predict Wee1 sensitivity with an 85% of prediction accuracy. The data support the hypothesis that, given the frequent deregulation and high prediction accuracy by the two genes, the expression pattern/ratio of MAD1 to SMAD2 will find use in predicting a patient's potential response, e.g., sensitivity to a combination therapy comprising a Wee1 inhibitor and a DNA damaging agent.

1-39. (canceled)

40. A method for predicting a patient's response to a treatment with a Wee1 inhibitor comprising:
(a) calculating the measure of similarity between (i) a patient gene expression profile and a Wee1 inhibitor responder template, or (ii) a patient gene expression profile and a Wee1 inhibitor non-responder template, or (iii) a patient gene expression profile and both a Wee1 inhibitor responder and non-responder template; and
(b) predicting said patient's response to said treatment from said measure of similarity;
wherein (i) a patient gene expression profile comprises measuring the nucleic acid expression level of each biomarker gene in a biological sample obtained from said patient, (ii) said Wee1 inhibitor responder template comprises measuring the average nucleic acid expression level of each biomarker gene obtained from a plurality of control cell samples that are sensitive to said Wee1 inhibitor, (iii) said Wee1 inhibitor non-responder template comprises measuring the average nucleic acid expression level of each biomarker gene obtained from a plurality of control cell samples that are resistant to said Wee1 inhibitor, and (iv) said biomarker gene comprises one or more genes selected from the group consisting of SPRY2, CCNI, JUNB, SMAD2, SHC1, MAD1L1, GADD45GIP1, CKAP5, TUBB4, BCAT1, MCM8 and TLK2.

41. The method according to claim 40, wherein said patient response comprises:
(a) a favorable response to said treatment protocol if said patient gene expression profile has a high similarity to said Wee1 inhibitor responder template or has a higher similarity to said Wee1 inhibitor responder template than to said Wee1 inhibitor non-responder template; and
(b) an unfavorable response to said treatment protocol if said patient gene expression profile has low similarity to said Wee1 inhibitor responder template or a higher similarity to said Wee1 inhibitor non-responder template than to said Wee1 inhibitor responder template;
wherein said patient gene expression profile has a high similarity to said Wee1 inhibitor responder template if the similarity to said Wee1 inhibitor responder template is above a predetermined threshold, or has a low similarity to said Wee1 inhibitor responder template if the similarity to said Wee1 inhibitor responder template is below said predetermined threshold.

42. The method according to claim 40, wherein said biomarker genes are one of MAD1L1 and SMAD2.

43. The method according to claim 40, wherein a change in the expression level of said biomarker gene obtained from a patient is at least 1.5 fold or greater relative to that obtained from a control cell sample.

44. The method according to claim 40, wherein said biomarker genes are a combination of at least one of the group consisting of SPRY2, MAD1L1, GADD45GIP1, CKAP5, TUBB4, BCAT1, MCM8 and TLK2 and at least one of the group consisting of CCNI, JUNB, SMAD2 and SHC1.

45. The method according to claim 40, wherein said Wee1 inhibitor template is derived using the Wee1 inhibitor, 2-allyl-1-[6-(1-hydroxy-1-methylethyl)pyridin-2-yl]-6-[({4-(4-methylpiperazin-1-yl)phenyl]amino}-1,2-dihydro-3H-pyrazolo[3,4-d]pyrimidin-3-one.

46. A method for predicting the response of a patient diagnosed with a cancer to treatment with a Wee 1 inhibitor comprising:
(a) measuring the gene expression level of each gene of a Wee1 expression profile comprising one or more prediction biomarker genes selected from the group consisting of SPRY2, CCNI, JUNB, SMAD2, SHC1, MAD1L1, GADD45GIP1, CKAP5, TUBB4, BCAT1, MCM8 and TLK2 in a biological sample comprising cancer cells obtained from said patient;
(b) obtaining a cumulative gene expression measurement for said expression profile by summing up the gene expression level for each biomarker gene; and
(c) determining whether the cumulative gene expression measurement is above or below a pre-determined threshold;
wherein a cumulative gene expression measurement above or below said pre-determined threshold is predictive of the patient's treatment response to a Wee 1 inhibitor.

47. The method according to claim 46, wherein said Wee 1 expression profile comprises measuring a plurality of genes selected from the group consisting of SPRY2, CCNI, JUNB, SMAD2, SHC1, MAD1L1, GADD45GIP1, CKAP5, TUBB4, BCAT1, MCM8 and TLK2 in a biological sample comprising cancer cells obtained from said patient to determine a mean average expression level, wherein mean average expression level above or below a pre-determined threshold is predictive of the patient's treatment response to said Wee1 inhibitor.

48. The method according to claim 47, wherein the pre-determined threshold is at least 1 to 2 fold over-expressed in the cancer patient sample relative to that from a non-cancerous patient sample.

49. The method according to claim 47, wherein the pre-determined threshold has at least a statistically significant p-value over expression in the cancer patient sample relative to a sample from a non-cancerous or a normal patient, or to a sample from a patient not exhibiting aberrant Wee1 signaling.

50. The method according to claim 49, wherein the p-value is less than 0.05.

51. The method according to claim 47, wherein said pre-determined threshold is the average of each of said plurality of genes in a sample obtained from a disease subject or a subject whose cells do not exhibit aberrant Wee1 signaling.

52. The method according to claim 46, wherein said patient has been diagnosed with a Wee1 mediated cancer, said Wee 1 expression profile comprises measuring a plurality of genes selected from the group consisting of SPRY2, MAD1L1, GADD45GIP1, CKAP5, TUBB4, BCAT1, MCM8 and TLK2 to determine an average expression level for each gene, and an increase in said average expression level relative to a pre-determined threshold is predictive of the patient's treatment response to the Wee 1 inhibitor.

53. The method according to claim 52, wherein said Wee1 expression profile comprises measuring a plurality of genes selected from the group consisting of CCNI, JUNB, SMAD2 and SHC1, and a decrease in said average expression level relative to a pre-determined threshold is predictive of the patient's treatment response to the Wee 1 inhibitor.

54. The method according to claim 52, wherein the pre-determined threshold is at least 1 to 2 fold over-expressed in the cancer patient sample relative to that from a non-cancerous patient sample.

55. The method according to claim 52, wherein the pre-determined threshold has at least a statistically significant p-value over expression in the cancer patient sample relative to a sample from a non-cancerous or a normal patient, or to a sample from a patient not exhibiting aberrant Wee1 signaling.

56. The method according to claim 52, wherein the p-value is less than 0.05.

57. The method according to claim 52, wherein said pre-determined threshold is the average level of expression of each of said genes across a plurality of control samples derived from disease free subjects.

58. A method for stratifying a patient diagnosed with a cancer responsive to treatment with a Wee1 inhibitor responsive for a clinical trial comprising:
(a) measuring the gene expression for one or more Wee1 biomarker genes in a clinical sample of diseased cells obtained from a cancer patient;
(b) comparing the measured gene expression for each Wee1 biomarker gene in said clinical sample with the gene expression for the same one or more Wee1 biomarker genes in a control sample; and
(c) stratifying the patient for the clinical trial based on the results of step (b); wherein said one or more Wee1 biomarker genes are selected from the group consisting of SPRY2, CCNI, JUNB, SMAD2, SHC1, MAD1L1, GADD45GIP1, CKAP5, TUBB4, BCAT1, MCM8 and TLK2.

59. The method according to claim 56, wherein the measuring step comprises detecting mRNA expression levels of said Wee1 biomarker genes.

60. A method for predicting whether a patient diagnosed with a Wee1 mediated cellular proliferative disorder is likely to respond to a Wee1 inhibitor based therapy comprising:
(a) calculating the measure of similarity between (i) a patient gene expression profile and a Wee1 inhibitor responder template, or (ii) a patient gene expression profile and a Wee1 inhibitor non-responder template, or (iii) a patient gene expression profile and both a Wee1 inhibitor responder and non-responder template; and
(b) predicting said patient's response to said treatment from said measure of similarity;
wherein (i) a patient gene expression profile comprises measuring the nucleic acid expression level of each biomarker gene in a biological sample obtained from said patient, (ii) said Wee1 inhibitor responder template comprises measuring the average nucleic acid expression level of each biomarker gene obtained from a plurality of control cell samples that are sensitive to said Wee1 inhibitor, (iii) said Wee1 inhibitor non-responder template comprises measuring the average nucleic acid expression level of each biomarker gene obtained from a plurality of control cell samples that are resistant to said Wee1 inhibitor, and (iv) said biomarker gene comprises one or more genes selected from the group consisting of SPRY2, CCNI, JUNB, SMAD2, SHC1, MAD1L1, GADD45GIP1, CKAP5, TUBB4, BCAT1, MCM8 and TLK2.

61. The method according to claim 60, wherein said patient response comprises:
(a) is predicted to be sensitive to Wee1 inhibitor treatment if said patient gene expression profile has a high similarity to said Wee1 inhibitor responder template or has a higher similarity to said Wee1 inhibitor responder template than to said Wee1 inhibitor non-responder template; and
(b) is predicted to be resistant to Wee1 inhibitor treatment if said patient gene expression profile has low similarity to said Wee1 inhibitor responder template or a higher similarity to said Wee1 inhibitor non-responder template than to said Wee1 inhibitor responder template;
wherein said patient gene expression profile has a high similarity to said Wee1 inhibitor responder template if the similarity to said Wee1 inhibitor responder template is above a predetermined threshold, or has a low similarity to said Wee1 inhibitor responder template if the similarity to said Wee1 inhibitor responder template is below said predetermined threshold.

62. The method according to claim 60, wherein said control is the average gene expression of said plurality of genes obtained from a disease free subject or a subject whose cells do not exhibit aberrant Wee1 signaling.

63. The method according to claim 60, wherein an increase in the average gene expression in the patient sample relative to a control sample indicates that the patient is more likely to respond to treatment with the Wee1 inhibitor.

64. The method according to claim 61, further comprising treating a patient predicted to be sensitive to Wee1 inhibitor treatment with a Wee1 inhibitor.

65. The method according to claim 61, further comprising pulling out patients predicted to be resistant to Wee1 inhibitor.

66. The method according to claim 64, wherein the Wee1 inhibitor is 2-allyl-1-[6-(1-hydroxy-1-methylethyl)pyridin-2-yl]-6-{[4-(4-methylpiperazin-1-yl)phenyl]amino}-1,2-dihydro-3H-pyrazolo[3,4-d]pyrimidin-3-one.
